(19) United States
(12) Patent Application Publication
(10) Pub. No.: US 2008/0038295 A1
(43) Pub. Date: Feb. 14, 2008

(54) Compositions and methods for orthopox virus vaccination

(75) Inventors: James R. Baker JR., Ann Arbor, MI (US); Anna Bielinska, Ypsilanti, MI (US); Andrzej Myc, Ann Arbor, MI (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/786,860

(22) Filed: Apr. 13, 2007

Related U.S. Application Data
(60) Provisional application No. 60/791,800, filed on Apr. 13, 2006.

(51) Int. Cl. A61K 39/275; A61P 31/00

(52) U.S. Cl. 424/232.1

(57) ABSTRACT

The present invention relates to methods and compositions for stimulating an immune response. Specifically, the present invention provides methods of inducing an immune response to an orthopox virus (e.g., vaccinia virus) in a subject (e.g., a human subject) and compositions useful in such methods (e.g., a nanoemulsion comprising vaccinia virus). Compositions and methods of the present invention find use in, among other things, clinical (e.g. therapeutic and preventative medicine (e.g., vaccination) and research applications.

The present invention claims priority to U.S. Provisional Patent Application Ser. No. 60/791,800 filed Apr. 13, 2006, hereby incorporated by reference in its entirety.

This invention was made with government support under contract U54 AI57153-02 awarded by the National Institutes of Health and contract MDA972-97-1-0007 awarded by the Department of Defense-Defense Advanced Research Projects Agency. The government has certain rights in the invention.

### FIELD OF THE INVENTION

The present invention relates to methods and compositions for stimulating an immune response. Specifically, the present invention provides methods of inducing an immune response to an orthopox virus (e.g., vaccinia virus) in a subject (e.g., a human subject) and compositions useful in such methods (e.g., a nanoemulsion comprising vaccinia virus). Compositions and methods of the present invention find use in, among other things, clinical (e.g. therapeutic and preventative medicine (e.g., vaccination)) and research applications.

### BACKGROUND OF THE INVENTION

Smallpox was once considered the most dreaded human disease, killing between 300 and 540 million people in the 20^{th} century alone (See, e.g., Selgelid, J Med Ethics. December 2004;30(6):558-60). Due to aggressive efforts of the World Health Organization's vaccination program, small pox was declared eradicated in 1980 and the WHO recommended that all countries cease vaccination (See, e.g., Selgelid, J Med Ethics. December 2004;30(6):558-60). Termination of compulsory smallpox vaccination was welcomed because the smallpox vaccine, a live virus vaccine (Vaccinia virus (VV)), was associated with known mild to severe adverse effects, including inadvertent inoculation and mortality. In 1976, the WHO also asked research laboratories with the smallpox virus to destroy their stocks, and while most laboratories complied with the WHO's request, there has been evidence that some nations have programs to develop smallpox as a biological weapon (See, e.g., Riedel, S. Proc (Bayl Univ Med Cent). January 2005; 18(1): 13-20). This evidence has initiated a political and ethical debate regarding the reintroduction of the smallpox vaccination (See, e.g., Poland et al., Vaccine 23 (2005) 2078-2081).

Following Sep. 11, 2001, the United States only had 15.4 million doses of smallpox vaccine (Dryvax, Wyeth Laboratories) and 80 million doses of small pox vaccine (Wetvax) donated by Aventis Pasteur (Paris, France). In addition to possessing a finite and numerically inadequate stockpile of vaccines, both vaccines were manufactured over 20 years ago and were produced according to safety standards that do not meet those of vaccines routinely used today (See, e.g., Bonilla-Guerrero and Poland, J. of Laboratory and Clinical Medicine 142 (2003), 252-257). Both Dryvax (last produced by Wyeth Laboratories 20 years ago) and Wetvax (in storage for 40 years) vaccines are formulation of live Vaccinia virus. In addition, new vaccine stockpiles produced or conceived after 2001 have been developed in similar manner, providing live virus vaccines (See, e.g., Grabenstein and Winkenwerder Jr., JAMA 289 (2003) (24), pp. 3278-3282). The vaccination program undertaken before the Iraq war is demonstrative of the unwanted, and unavoidable, adverse risks and effects that are associated with the current smallpox vaccines of choice (See, e.g., Poland et al., Vaccine 23 (2005) 2078-2081; Bonilla-Guerrero and Poland, J. of Laboratory and Clinical Medicine 142 (2003), 252-257).

Several formulations of killed Vaccinia virus vaccines exist. However, these formulations suffer from a lack of efficacy (e.g., poor immunogenicity elicited in a subject) and generally require co-administration with adjuvants that may result in inflammation and autoimmunity (See, e.g., Earl et al., Nature 428 (2004) (6979), pp. 182-185; Vollmar et al., Vaccine 24 (2006) 2065-2070; Coulibaly et al., Virology. Oct. 10, 2005;341(1):91-101; Frey et al., N Engl J Med 346 (2002) (17), pp. 1265-1274).

What is needed is a new smallpox vaccine that retains the efficacy of existing live vaccines that does not generate adverse effects (e.g., morbidity and mortality) when administered to a subject.

### SUMMARY OF THE INVENTION

The present invention relates to methods and compositions for stimulating an immune response. Specifically, the present invention provides methods of inducing an immune response to an orthopox virus (e.g., vaccinia virus) in a subject (e.g., a human subject) and compositions useful in such methods (e.g., a nanoemulsion comprising vaccinia virus). Compositions and methods of the present invention find use in, among other things, clinical (e.g. therapeutic and preventative medicine (e.g., vaccination) and research applications.

Accordingly, in some embodiments, the present invention provides a method of inducing an immune response to an orthopox virus in a subject comprising: providing a composition comprising a nanoemulsion and an immunogen, wherein the immunogen comprises orthopox virus inactivated by the nanoemulsion; and administering the composition to the subject under conditions such that the subject generates an immune response to the orthopox virus. The present invention is not limited by the nature of the immune response generated. Indeed, a variety of immune responses may be generated and measured in a subject administered a composition comprising a nanoemulsion and an immunogen of the present invention including, but not limited to, activation, proliferation or differentiation of cells of the immune system (e.g., B cells, T cells, dendritic cells, antigen presenting cells (APCs), macrophages, natural killer (NK) cells, etc.); up-regulated or down-regulated expression of markers and cytokines; stimulation of IgA, IgM, or IgG titer; splenomegaly (e.g., increased spleen cellularity); hyperplasia, mixed cellular infiltrates in various organs, and other responses (e.g., of cells) of the immune system that can be assessed with respect to immune stimulation known in the art. In some embodiments, administering comprises contacting a mucosal surface of the subject with the composition. The present invention is not limited by the mucosal surface contacted. In some preferred embodiments, the mucosal surface comprises nasal mucosa. In some embodiments, administrating comprises parenteral administration. The present invention is not limited by the route chosen for administration of a composition of the present invention. In some embodiments, inducing an immune response induces immunity to the orthopox virus in the subject. In some embodiments, the immunity comprises systemic immunity. In some embodiments, the immunity comprises mucosal immunity. In some embodiments, the immune response comprises increased expression of IFN-γ in the subject. In some embodiments, the immune response comprises a systemic IgG response to the inactivated orthopox virus. In some embodiments, the immune response comprises a mucosal IgA response to the inactivated orthopox virus. The present invention is not limited by the type of orthopox virus used in a composition of the present invention. Indeed, a variety of orthopox viruses may be used including, but not limited to, variola virus, vaccinia virus, cowpox, monkeypox, gerbilpox, camelpox, among others. In some embodiments, the orthopox virus inactivated by the nanoemulsion is administered to the subject under conditions such that between 10 and 10³ pfu of the inactivated virus is present in a dose administered to the subject. However, the present invention is not limited to this amount of orthopox virus administered. For example, in some embodiments, more than 10³ pfu of the inactivated virus (e.g., 10⁴ pfu, 10⁵ pfu, or more) is present in a dose administered to the subject. In some embodiments, a 10% nanoemulsion solution is utilized to inactivate the orthopox virus. However, the present invention is not limited to this amount (e.g., percentage) of nanoemulsion used to inactivate a orthopox virus. For example, in some embodiments, a composition comprising less than 10% nanoemulsion is used for inactivation. In some embodiments, a composition comprising more than 10% nanoemulsion is used for inactivation. In some embodiments, the nanoemulsion comprises W₂₀5EC. The present invention is not limited by the type of nanoemulsion utilized. Indeed, a variety of nanoemulsions are contemplated to be useful in the present invention. For example, in some preferred embodiments, the nanoemulsion (e.g., for generating an immune response (e.g., for use as a vaccine)) comprises an oil-in-water emulsion, the oil-in-water emulsion comprising a discontinuous oil phase distributed in an aqueous phase, a first component comprising a solvent (e.g., an alcohol or glycerol), and a second component comprising a surfactant or a halogen-containing compound. The aqueous phase can comprise any type of aqueous phase including, but not limited to, water (e.g., diH₂O, distilled water, tap water) and solutions (e.g., phosphate buffered saline solution). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In some preferred embodiments, the oil phase comprises 30-90 vol % of the oil-in-water emulsion (i.e., constitutes 30-90% of the total volume of the final emulsion), more preferably 50-80%. While the present invention in not limited by the nature of the alcohol component, in some preferred embodiments, the alcohol is ethanol or methanol. Furthermore, while the present invention is not limited by the nature of the surfactant, in some preferred embodiments, the surfactant is a polysorbate surfactant (e.g., TWEEN 20, TWEEN 40, TWEEN 60, and TWEEN 80), a pheoxypolyethoxyethanol (e.g., TRITON X-100, X-301, X-165, X-102, and X-200, and TYLOXAPOL) or sodium dodecyl sulfate. Likewise, while the present invention is not limited by the nature of the halogen-containing compound, in some preferred embodiments, the halogen-containing compound comprises a cetylpyridinium halides, cetyltrimethylammonium halides, cetyldimethylethylammonium halides, cetyldimethylbenzylammonium halides, cetyltributylphosphonium halides, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyidimethylethylammonium bromide, cetyltributylphosphonium bromide, dodecyltrimethylammonium bromide, or tetrad ecyltrimethylammonium bromide. Nanoemulsions of the present invention may further comprise third, fourth, fifth, etc. components. In some preferred embodiments, an additional component is a surfactant (e.g., a second surfactant), a germination enhancer, a phosphate based solvent (e.g., tributyl phosphate), a neutramingen, L-alanine, ammonium chloride, trypticase soy broth, yeast extract, L-ascorbic acid, lecithin, p-hyroxybenzoic acid methyl ester, sodium thiosulate, sodium citrate, inosine, sodium hyroxide, dextrose, and polyethylene glycol (e.g., PEG 200, PEG 2000, etc.). In some embodiments, the oil-in-water emulsion comprises a quaternary ammonium compound. In some preferred embodiments, the oil-in-water emulsion has no detectable toxicity to plants or animals (e.g., to humans). In other preferred embodiments, the oil-in-water emulsion causes no detectable irritation to plants or animals (e.g., to humans). In some embodiments, the oil-in-water emulsion further comprises any of the components described above. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N, N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C1 6); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis (2-hydroxyethyl) octyl hydrogen ammonium chloride; dodecyl dimethyl benzyl ammonium chloride; dodecylcarbamoyl methyl dinethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride. In some embodiments, the emulsion lacks any antimicrobial substances (i.e., the only antimicrobial composition is the emulsion itself). In some embodiments, the nanoemulsion is X8P. In some embodiments, the immunity protects the subject from displaying signs or symptoms of disease caused by an orthopox virus (e.g., vaccinia virus). In some embodiments, the immunity protects the subject from challenge with a subsequent exposure to live orthopox virus. In some embodiments, induction of an immune response protects a subject from morbidity and/or mortality associated with orthopox virus infection. In some embodiments, the composition further comprises an adjuvant. The present invention is not limited by the type of adjuvant utilized. A number of adjuvants that find use in the present invention are described herein. In some embodiments, the subject is a human.

The present invention also provides a composition for stimulating an immune response comprising a nanoemulsion and an orthopox virus inactivated by the nanoemulsion, wherein the composition is configured to induce immunity to the orthopox virus in a subject. In some embodiments, the nanoemulsion comprises W₂₀5EC. In some embodiments, the composition provides a subject between 10 and 10³ pfu of the inactivated virus when administered to the subject. In some embodiments, the composition provides a subject between 10³ and 10⁵ pfu of the inactivated virus when administered to the subject. In some embodiments, a dose of the composition that is administered to a subject comprises a 1% nanoemulsion solution. In some embodiments, the inactivated orthopox virus is heat stable in the nanoemulsion. In some embodiments, the orthopox virus is stable for greater than four weeks in the nanoemulsion. In some embodiments, the orthopox virus is vaccinia virus. The present invention is not limited by the type of orthopox virus used. Indeed, a variety of orthopox viruses can be used in a composition for stimulating an immune response including, but not limited to, variola virus, cowpox, monkeypox, gerbilpox, and camelpox. In some embodiments, the composition is diluted prior to administration to a subject. In some embodiments, the subject is a human. In some embodiments, the immunity is systemic immunity. In some embodiments, the immunity is mucosal immunity. In some embodiments, the composition further comprises an adjuvant.

The present invention also provides a kit comprising a composition for stimulating an immune response comprising a nanoemulsion and an orthopox virus inactivated by the nanoemulsion, wherein the composition is configured to induce immunity to the orthopox virus in a subject, and instructions for administering the composition.

### DESCRIPTION OF THE DRAWINGS

FIG. 1 shows complete virus inactivation with nanoemulsion. (A) Plaque reduction assay (PRA) of VV_{WR}. (B) Luciferase assay of VV_{WR-Luc}. Luciferase activity is presented in relative light units (RLU). (C) PCR analysis of lung DNA. Lane 1: DNA size marker; lane 2: primers, no DNA; lane 3: no Taq; lane 4: 10⁵/Fk lung DNA; lanes 5-7: 10⁵/Fk/NE lung DNA; lanes 8-10: 10⁵/NE lung DNA; lane 11: control—VV DNA mixed with lung DNA. Arrows indicate amplified viral template and primers. (D) In vivo bioluminescence imaging of mice after intranasal infection with live VV_{WR-Luc} and with 10⁵ pfu of NE-killed virus. Circles visible in some images indicate region-of-interest (ROI) for photon flux analysis.

FIG. 2 shows immunogenicity of mucosal nanoemulsion vaccine in mice. (A) Development of serum anti-VV IgG antibody response in mice vaccinated with various formulations of killed virus vaccine: 10⁵/NE (filled circle), 10³/NE (open circle), 10⁵/Fk/NE (filled triangle), 10³/Fk/NE (open triangle), 10⁵/NFk (filled diamond) and 10³/Fk (open diamond). Arrows indicate i.n administrations of the vaccine. Insert: Comparison of serum anti-VV IgG after one or three vaccinations with 10⁵/NE vaccine. Data presented as mean of the individual anti-VV IgG concentrations±sem. (B) Secretory anti-VV IgA antibody in BAL. Results are presented as mean concentrations (±sem) of IgA obtained in assays performed with individual and pooled BAL fluids.

FIG. 3 shows virus neutralizing antibodies. Assays were performed with both individual and with pooled sera obtained after one, two and three vaccinations. Insert: Detection of virus neutralizing activity in BAL. Assays were performed with individual and pooled BAL fluids collected at the conclusion of the experiment at 16 weeks. Results were normalized and presented as NT₅₀ of the viral PRA.

FIG. 4 shows vaccinia-specific cellular immune responses. The INF-γ expression in vitro in splenocytes stimulated with 10³ and 10⁴ pfu of live VV_{WR}. The data show a specific INF-γ response to the virus in splenocytes from animals immunized with vaccinia virus inactivated by NE.

FIG. 5 shows intranasal challenge with live vaccine virus. (A) Survival curves for mice vaccinated with 10⁵ pfu of killed VV_{WR} in various vaccine formulations: VV/NE, VV/Fk/NE and VV/Fk, after i.n. challenge with 10×LD₅₀ VV_{WR-Luc}. (B) Bioluminescence images of representative vaccinated (upper panel) and control mouse (lower panel). Images were recorded 2 to 5 days after challenge.

### DEFINITIONS

To facilitate an understanding of the present invention, a number of terms and phrases are defined below:

As used herein, the term “microorganism” refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

As used herein the term “pathogen,” and grammatical equivalents, refers to an organism (e.g., biological agent), including microorganisms, that causes a disease state (e.g., infection, pathologic condition, disease, etc.) in another organism (e.g., animals and plants) by directly infecting the other organism, or by producing agents that causes disease in another organism (e.g., bacteria that produce pathogenic toxins and the like). “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc.

As used herein, the term “fungi” is used in reference to eukaryotic organisms such as molds and yeasts, including dimorphic fungi.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

The terms “host” or “subject,” as used herein, refer to an individual to be treated by (e.g., administered) the compositions and methods of the present invention. Subjects include, but are not limited to, mammals (e.g., murines, simians, equines, bovines, porcines, canines, felines, and the like), and most preferably includes humans. In the context of the invention, the term “subject” generally refers to an individual who will be administered or who has been administered one or more compositions of the present invention (e.g., a composition for inducing an immune response).

As used herein, the terms “inactivating,” “inactivation” and grammatical equivalents, when used in reference to a microorganism (e.g., a pathogen (e.g., a orthopox virus (e.g., vaccinia virus))), refer to the killing, elimination, neutralization and/or reducing of the capacity of the mircroorganism (e.g., a pathogen (e.g., orthopox virus (e.g., vaccinia virus))) to infect and/or cause a pathological response and/or disease in a host. In some preferred embodiments, the present invention provides a composition comprising nanoemulsion (NE)-inactivated orthopox virus (e.g., vaccinia virus (VV)). Accordingly, as referred to herein, compositions comprising “NE-inactivated orthopox virus,” “NE-killed orthopox virus,” NE-neutralized orthopox virus” or grammatical equivalents refer to compositions that, when administered to a subject, are characterized by the absence of, or significantly reduced presence of, orthopox virus replication (e.g., over a period of time (e.g., over a period of days, weeks, months, or longer)) within the host.

As used herein, the term “fusigenic” is intended to refer to an emulsion that is capable of fusing with the membrane of a microbial agent (e.g., a bacterium or bacterial spore). Specific examples of fusigenic emulsions include, but are not limited to, W₂₀5EC (See, e.g., Example 1), W₈₀8P, those described in U.S. Pat. Nos. 5,618,840; 5,547,677; and 5,549,901 and NP9 described in U.S. Pat. No. 5,700,679, each of which is herein incorporated by reference in their entireties. NP9 is a branched poly(oxy-1,2 ethaneolyl),alpha-(4-nonylphenal)-omega-hydroxy-surfactant. While not being limited to the following, NP9 and other surfactants that may be useful in the present invention are described in Table 1 of U.S. Pat. No. 5,662,957, herein incorporated by reference in its entirety.

As used herein, the term “lysogenic” refers to an emulsion (e.g., a nanoemulsion) that is capable of disrupting the membrane of a microbial agent (e.g., a virus (e.g., viral envelope) or a bacterium or bacterial spore). In preferred embodiments of the present invention, the presence of a lysogenic and a fusigenic agent in the same composition produces an enhanced inactivating effect compared to either agent alone. Methods and compositions (e.g., for inducing an immune response (e.g., used as a vaccine) using this improved antimicrobial composition are described in detail herein.

The term “emulsion,” as used herein, includes classic oil-in-water or water in oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in preferred embodiments, the nanoemulsions comprise an oil phase having droplets with a mean particle size of approximately 0.1 to 5 microns (e.g., 150±25 nm in diameter), although smaller and larger particle sizes are contemplated. The terms “emulsion” and “nanoemulsion” are often used herein, interchangeably, to refer to the nanoemulsions of the present invention.

As used herein, the terms “contact,” “contacted,” “expose,” and “exposed,” when used in reference to a nanoemulsion and a live microorganism, refer to bringing one or more nanoemulsions into contact with a microorganism (e.g., pathogen (e.g., a virus)) such that the nanoemulsion inactivates the microorganism or pathogenic agent, if present. The present invention is not limited by the amount or type of nanoemulsion used for microorganism inactivation. A variety of nanoemulsions that find use in the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes). Ratios and amounts of nanoemulsion (e.g., sufficient for inactivating the microorganism (e.g., virus inactivation)) and microorganisms (e.g., sufficient to provide an antigenic composition (e.g., a composition capable of inducing an immune response)) are contemplated in the present invention including, but not limited to, those described herein (e.g., in Example 1).

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described, for example, by Meyers, (See, e.g., Meyers, Surfactant Science and Technology, VCH Publishers Inc., New York, pp. 231-245 (1992)), incorporated herein by reference. As used herein where appropriate, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water that are good solubilizers of water in oils are at the low end of the scale.

As used herein the term “interaction enhancers” refers to compounds that act to enhance the interaction of an emulsion with a microorganism (e.g., with a cell wall of a bacteria (e.g., a Gram negative bacteria) or with a viral envelope (e.g., Vaccinia virus envelope)). Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum abulmin (BSA) and the like).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “a composition for inducing an immune response” refers to a composition that, once administered to a subject (e.g., once, twice, three times or more (e.g., separated by weeks, months or years)), stimulates, generates and/or elicits an immune response in the subject (e.g., resulting in total or partial immunity to a microorganism (e.g., pathogen) capable of causing disease). In preferred embodiments of the invention, the composition comprises a nanoemulsion and an immunogen (e.g., wherein the immunogen comprises an orthopox virus (e.g., Vaccinia virus) inactivated by the nanoemulsion, or, one or more orthopox virus antigens (e.g., purified (e.g., synthetic, recombinant or otherwise isolated) proteins or derivatives or analogues thereof). In further preferred embodiments, the composition comprising a nanoemulsion and an immunogen comprises one or more other compounds or agents including, but not limited to, therapeutic agents, physiologically tolerable liquids, gels, carriers, diluents, adjuvants, excipients, salicylates, steroids, immunosuppressants, immunostimulants, antibodies, cytokines, antibiotics, binders, fillers, preservatives, stabilizing agents, emulsifiers, and/or buffers. An immune response may be an innate (e.g., a non-specific) immune response or a learned (e.g., acquired) immune response (e.g. that decreases the infectivity, morbidity, or onset of mortality in a subject (e.g., caused by exposure to a pathogenic microorganism) or that prevents infectivity, pathology, morbidity, or onset of mortality in a subject (e.g., caused by exposure to a pathogenic microorganism)). Thus, in some preferred embodiments, a composition comprising a nanoemulsion and an immunogen (e.g., inactivated Vaccinia virus) is administered to a subject as a vaccine (e.g., to prevent or attenuate a disease (e.g., by providing to the subject total or partial immunity against the disease or the total or partial attenuation (e.g., suppression) of a sign, symptom or condition of the disease)).

As used herein, the term “adjuvant” refers to any substance that can stimulate an immune response. Some adjuvants can cause activation of a cell of the immune system (e.g., an adjuvant can cause an immune cell to produce and secrete a cytokine).

As used herein, the terms “an amount effective to induce an immune response” and “effective amount” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any condition that leads to a qualitative or quantitative induction, generation, and/or stimulation of an immune response (e.g., innate or acquired).

A used herein, the term “immune response” refers to any detectable response by the immune system of a subject. For example, immune responses include, but are not limited to, an alteration (e.g., increase) in Toll receptor activation, lymphokine (e.g., cytokine (e.g., Th1 or Th2 type cytokines) or chemokine) expression and/or secretion, macrophage activation, dendritic cell activation, T cell (e.g., CD4+ or CD8+ T cell) activation, NK cell activation, and/or B cell activation (e.g., antibody generation and/or secretion). Additional examples of immune responses include binding of an immunogen (e.g., antigen (e.g., immunogenic polypeptide)) to an MHC molecule and induction of a cytotoxic T lymphocyte (“CTL”) response, induction of a B cell response (e.g., antibody production), and/or T-helper lymphocyte response, and/or a delayed type hypersensitivity (DTH) response (e.g., against the antigen from which an immunogenic polypeptide is derived), expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to an antigen and/or immunogen (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression of) a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

As used herein, the term “immunogen” refers to an agent (e.g., a microorganism (e.g., bacterium, virus or fungus) or portion thereof (e.g., an antigen (e.g., gp120 or rPA))) that is capable of eliciting an immune response in a subject. In preferred embodiments, immunogens elicit immunity against the immunogen (e.g., microorganism (e.g., pathogen) or portion thereof (e.g., an antigen (e.g., gp120 or rPA))) when administered in combination with a nanoemulsion of the present invention.

As used herein, the term “pathogen product” refers to any component or product derived from a pathogen including, but not limited to, polypeptides, peptides, proteins, nucleic acids, membrane fractions, and polysaccharides.

As used herein, the term “enhanced immunity” refers to an increase in the level of adaptive and/or acquired immunity in)a subject to a given immunogen (e.g., microorganism (e.g., pathogen)) following administration of a composition (e.g., composition for inducing an immune response of the present invention) relative to the level of adaptive and/or acquired immunity in a subject that has not been administered the composition (e.g., composition for inducing an immune response of the present invention).

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

As used herein, the terms “administration” and “administering” refer to the act of giving a composition of the present invention (e.g., a composition for inducing an immune response (e.g., a composition comprising a nanoemulsion and an immunogen)) to a subject.

As used herein, the terms “co-administration” and “co-administering” refer to the administration of at least two agent(s) (e.g., a composition comprising a nanoemulsion and an immunogen and one or more other agents—e.g., an adjuvant) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. In other embodiments, co-administration is preferable to elicit an immune response in a subject to two or more different immunogens (e.g., microorganisms (e.g., pathogens)) at or near the same time (e.g., when a subject is unlikely to be available for subsequent administration).

As used herein, the term “topically” refers to application of a compositions of the present invention (e.g., a composition comprising a nanoemulsion and an immunogen) to the surface of the skin and/or mucosal cells and tissues (e.g., alveolar, buccal, lingual, masticatory, vaginal or nasal mucosa, and other tissues and cells which line hollow organs or body cavities).

In some embodiments, the compositions of the present invention are administered in the form of topical emulsions, injectable compositions, ingestible solutions, and the like. When the route is topical, the form may be, for example, a spray (e.g., a nasal spray), a cream, or other viscous solution (e.g., a composition comprising a nanoemulsion and an immunogen in polyethylene glycol).

The terms “pharmaceutically acceptable” or “pharmacologically acceptable,” as used herein, refer to compositions that do not substantially produce adverse reactions (e.g., toxic, allergic or pathological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk (e.g., a subject may be “at risk for disease” simply by being exposed to and interacting with other people that carry a risk of transmitting a pathogen), nor is it intended that the present invention be limited to any particular disease.

“Nasal application”, as used herein, means applied through the nose into the nasal or sinus passages or both. The application may, for example, be done by drops, sprays, mists, coatings or mixtures thereof applied to the nasal and sinus passages.

As used herein, the term “kit” refers to any delivery system for delivering materials. In the context of immunogenic agents (e.g., compositions comprising a nanoemulsion and an immunogen), such delivery systems include systems that allow for the storage, transport, or delivery of immunogenic agents and/or supporting materials (e.g., written instructions for using the materials, etc.) from one location to another. For example, kits include one or more enclosures (e.g., boxes) containing the relevant immunogenic agents (e.g., nanoemulsions) and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a nanoemulsion and an immunogen for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of an immunogenic agent needed for a particular use in a single container (e.g., in a single box housing each of the desired components). The term “kit” includes both fragmented and combined kits.

### DETAILED DESCRIPTION OF THE INVENTION

Several pathogenic microorganisms initiate infection by attaching to mucosal epithelial cells lining the gastro-intestinal, oropharyngeal, respiratory or genito-urinacy tracts. Some pathogens, such as influenza virus, Bordetella pertussis, or Vibrio cholerae, remain at or within the mucosal tissue, while others, such as Salmonella typhi or hepatitis A virus, possess mechanisms permitting penetration into deeper tissues and spread systemically. Specific and non-specific defense mechanisms of the mucous membranes provide first line protection against both types of pathogen. Non-specific effectors include, for example, resident macrophages, antimicrobial peptides, lactoferrin and lysozyme, extremes of pH, bile acids, digestive enzymes, mucus, shedding of epithelial cells, flushing mechanisms (peristalsis, ciliary beating, micturation, etc) and competition from local flora. However, successful pathogens have generally evolved means to survive the non-specific defenses present at the site they infect and it is the secretory immune system that plays a major role in protecting against diseases caused by a number of bacterial and viral pathogens, and is a major effector against pathogens that are restricted to mucosal surfaces. For organisms that spread systemically, both local and systemic immune responses are likely needed for optimum immunity.

Existing forms of orthopox (e.g., smallpox, cowpox, monkeypox, gerbilpox, camelpox, and others) vaccines are obsolete. For example, all current, licensed smallpox vaccines that were used to achieve smallpox eradication are based on live Vaccinia virus (VV) obtained from infected calf's skin and lymph nodes. Although these vaccines confer long-lasting immunity against several different orthopox viruses, the materials they are made from do not meet current standards for human vaccines. Furthermore, these vaccines also produce infectious skin pustules (pox) and infrequent but severe side reactions limiting their use in individuals (and their close contacts) with immunodeficiency, eczema, atopic dermatitis, or heart disease (See, e.g., Eichner and Schwehm, Epidemiology. 2004, 15(3):258-60).

It is anticipated that future applications for orthopox vaccines (e.g., smallpox, cowpox, monkeypox, gerbilpox, camelpox, and others) would be a response to either a bioterrorist attack or outbreaks of other orthopox infections, such as monkeypox or cowpox (See, e.g., Edghill-Smith et al., Nature Med. 2005, 11; 740-747). Because of this, the risk/benefit ratio of vaccination will require that new smallpox vaccines place a high priority on safety. Additionally, for use in emergent public health situations, it will be imperative to replace vaccines administered by scarification with a rapidly administered vaccine (e.g., a mucosal vaccine). Such administration, in addition to providing a vaccination and long-term immune protection, may provide a quick, on-site generator of immune responses (e.g., that reduce infection, symptoms and/or time course of disease).

Accordingly, the present invention provides methods of inducing an immune response to an orthopox viruse (e.g., vaccinia virus) in a subject (e.g., a human subject) and compositions useful in such methods (e.g., a nanoemulsion comprising an orthopox virus (e.g., vaccinia virus (VV))). In preferred embodiments, methods of inducing an immune response provided by the present invention are used for vaccination. Due to the rate of adverse events with existing orthopox (e.g., smallpox) vaccines, the present invention provides a significant improvement in orthopox (e.g., small pox) vaccination safety without compromising vaccine efficiency.

For example, the present invention describes the development of immunity (e.g., VV immunity) in a subject after mucosal administration (e.g., mucosal vaccination) of an inactivated orthopox virus (e.g., VV) preparation identified and characterized during development of the present invention. Nanoemulsion (NE), a surface-active antimicrobial material, was mixed with highly purified, cell culture-derived VV, resulting in a formulation (e.g., NE-killed VV composition) that is stable at room temperature (e.g., in some embodiments, for more than 2 weeks, more preferably more than 3 weeks, even more preferably more than 4 weeks, and most preferably for more than 5 weeks) and that can be used to induce an immune response against orthopox viruses (e.g., VV) in a subject (e.g., that can be used either alone or as an adjuvant for inducing an anti-VV immune response).

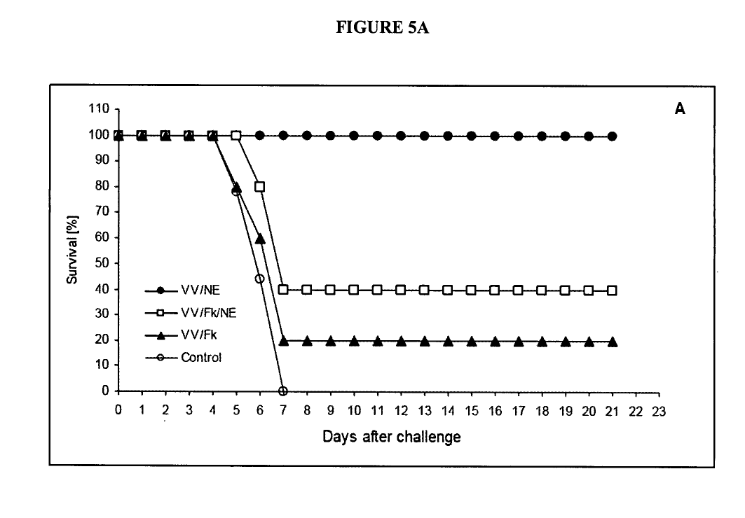

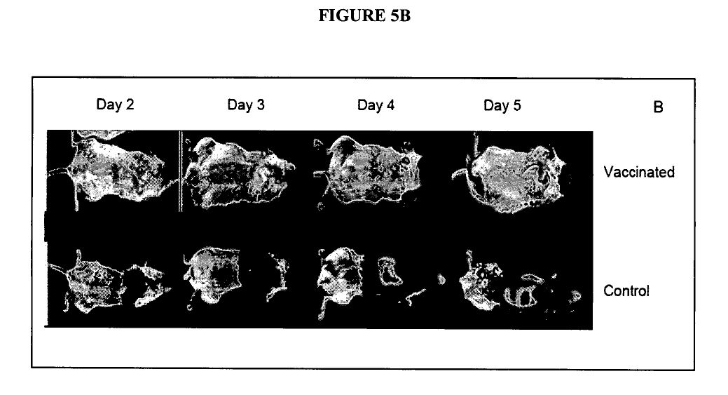

Mucosal administration of a composition comprising NE and VV (e.g., NE-killed VV) to a subject resulted in high-titer mucosal and systemic antibody responses and specific Th1 cellular immunity (See, e.g., Examples 2-4, and 6). Further, all animals were fully protected against an nasal instillation challenge with 10× LD₅₀ VV (See, e.g., Example 7, FIG. 5). In the vaccinated animals, infection was completely prevented or was of a low level and self-limiting and infection resolved in four to five days. In contrast, all naive animals died within this time period. Subsequent re-challenge of immunized mice with a 100× LD of VV-_{WR} validated protective immunity. Mice administered even a single dose of a composition comprising NE-killed VV developed significant serum concentrations of anti-VV IgG 10 to 12 weeks after administration (See, e.g., Example 2). This level of response is comparable to the results obtained in Balb/c mice immunized by intramuscular injection with live VV Wyeth at similar time point (See, e.g., Coulibaly et al., Virology, 2005; 341; 91-101). Thus, in some embodiments, the present invention provides that a single administration (e.g., mucosal administration) of a composition comprising NE-killed VV is sufficient to induce a protective immune response in a subject (e.g., protective immunity (e.g., mucosal and systemic immunity)). In some embodiments, a subsequent administration (e.g., one or more boost administrations subsequent to a primary administration) to a subject provides the induction of an enhanced immune response to VV in the subject. Thus, the present invention demonstrates that administration of a composition comprising NE-killed VV to a subject provides protective immunity against smallpox.

In contrast, intranasal instillations of formalin-killed VV with or without nanoemulsion produced inconsistent and low antibody responses, which did not augment even after a third immunization (See, e.g., Example 2). A similar pattern of neutralizing activity was also detected in serum and bronchoalveolar lavage (BAL), with neutralizing activity being absent in mice mucosally vaccinated with formalin-killed virus. Neutralizing activity was not detected in BAL of animals vaccinated with either IP or SQ injections of a live virus. Although an understanding of the mechanism is not necessary to practice the present invention and the present invention is not limited to any particular mechanism of action, in some embodiments, NE treatment (e.g., neutralization of an orthopox virus (e.g., VV) with a NE of the present invention) preserves important viral neutralizing epitopes (e.g., recognizable by a subject's immune system), stabilizing their hydrophobic and hydrophilic components in the oil and water interface of the emulsion (e.g., thereby providing one or more immunogens (e.g., stabilized antigens) against which a subject can mount an immune response). In other embodiments, because NE formulations can penetrate the mucosa through pores, they may carry viral proteins to the submucosal location of dendritic cells (e.g., thereby initiating and/or stimulating an immune response).

Dendritic cells avidly phagocytose NE oil droplets and this could provide a means to internalize antigenic proteins for antigen presentation. While other vaccines rely on inflammatory toxins or other immune stimuli for adjuvant activity (See, e.g., Holmgren and Czerkinsky, Nature Med. 2005, 11; 45-53), NEs have not been shown to be inflammatory when placed on the skin or mucous membranes in studies on animals and in humans. Thus, although an understanding of the mechanism is not necessary to practice the present invention and the present invention is not limited to any particular mechanism of action, in some embodiments, a composition comprising a NE of the present invention (e.g., a composition comprising NE-inactivated orthopox virus (e.g., VV)) may act as a “physical” adjuvant (e.g., that transports and/or presents orthopox antigens (e.g., Vaccina proteins) to the immune system. In some preferred embodiments, mucosal administration of a composition of the present invention generates mucosal as well as systemic immunity (e.g., signs of mucosal immunity (e.g., generation of IgA antibody titers).

Both cellular and humoral immunity play a role in protection against orthopoxviruses, and both were induced with the NE formulations (See, e.g., Examples 2-4, and 6). Vaccinia-specific antibody titers are considered important for the estimate of protective immunity in human subjects and in animal models of vaccination (See, e.g., Hammarlund et al, Nat. Med. 2003, 9; 1131-1137). Several studies have identified proteins important for the elicitation of neutralizing antibodies (See, e.g., Galmiche et al, Virology, 1999, 254; 71-80; Hooper et al, Virology, 2003, 306; 181-195). A recent trial of dilutions of the licensed smallpox vaccine (Dryvax) in human volunteers, confirmed that pustule formation strongly correlated with development of both specific antibodies and induction of cytotoxic T lymphocytes (CTL) and elevated INF-γ T cell responses (See, e.g., Greenberg et al, 2005, 365; 398-409). Induction of IFN-γ is suggestive of activation of specific MHC class I-restricted CD8+ T cells. These types of cells have been implicated in the recognition and clearance of Vaccinia infected cells, and for maintenance of immunity after vaccination (See, e.g., Earl et al, Nature, 2004; 482; 182-185; Hammarlund et al, Nat. Med. 2003, 9; 1131-1137; Edghill- Smith et all, Nature Med. 2005, 11; 740-747).

Thus, in some embodiments, administration (e.g., mucosal administration) of a composition of the present invention (e.g., NE-killed orthopox virus (e.g., VV) to a subject results in the induction of both humoral (e.g., development of specific antibodies) and cellular (e.g., cytotoxic T lymphocyte) immune responses (e.g., against the orthopox virus). In some preferred embodiments, a composition of the present invention (e.g., NE-killed orthopox virus (e.g., VV) is used as a smallpox vaccine.

Furthermore, in preferred embodiments, a composition of the present invention (e.g., NE-killed orthopox virus (e.g., VV) induces (e.g., when administered to a subject) both systemic and mucosal immunity. Thus, in some preferred embodiments, administration of a composition of the present invention to a subject results in protection against an exposure (e.g., a lethal mucosal exposure) to an orthopox virus (e.g., VV). Although an understanding of the mechanism is not necessary to practice the present invention and the present invention is not limited to any particular mechanism of action, mucosal administration (e.g., vaccination) provides protection against orthopox virus (e.g., VV) infection (e.g., that initiates at a mucosal surface). Although it has heretofore proven difficult to stimulate secretory IgA responses and protection against pathogens that invade at mucosal surfaces (See, e.g., Mestecky et al, Mucosal Immunology. 3ed edn. (Academic Press, San Diego, 2005)), the present invention provides compositions and methods for stimulating mucosal immunity (e.g., a protective IgA response) from a pathogen in a subject.

In some embodiments, the present invention provides a composition (e.g., a NE-inactivated orthopox virus (e.g., VV) formulation) to serve as a mucosal vaccine. This material can easily be produced (e.g., from purified virus (See, e.g., Example 1)), and induces both mucosal and systemic immunity (See, e.g., Examples 2-7). The ability to produce this formulation rapidly and administer it via nasal instillation provides a vaccine that can be used in large-scale outbreaks or emergent situations.

In some embodiments, the present invention provides compositions for generating an immune response and methods of using the same (e.g., for use as a vaccine). In some preferred embodiments, a composition for generating an immune response comprises a NE and an immunogen (e.g., an orthopox virus (e.g., VV) inactivated by the nanoemulsion). When administered to a subject, a composition of the present invention stimulates an immune response against the immunogen (e.g., orthopox virus (e.g., VV)) within the subject. Although an understanding of the mechanism is not necessary to practice the present invention and the present invention is not limited to any particular mechanism of action, in some embodiments, generation of an immune response (e.g., resulting from administration of a composition comprising a nanoemulsion and an orthopox virus (e.g., VV)) provides total or partial immunity to the subject (e.g., from signs, symptoms or conditions of a disease (e.g., smallpox)). Without being bound to any specific theory, protection and/or immunity from disease (e.g., the ability of a subject's immune system to prevent or attenuate (e.g., suppress) a sign, symptom or condition of disease) upon exposure to a nanoemulsion comprising an orthopox virus (e.g., VV) is due to adaptive (e.g., acquired) immune responses (e.g., immune responses mediated by B and T cells following exposure to a NE comprising an orthopox virus (e.g., VV) of the present invention (e.g., immune responses that exhibit increased specificity and reactivity to an orthopox virus (e.g., VV)).

In some embodiments, a NE comprising an immunogen (e.g., an orthopox virus (e.g., VV) inactivated by the NE) is administered alone. In some embodiments, a composition comprising a NE and an immunogen (e.g., an orthopox virus (e.g., VV) inactivated by the NE) comprises one or more other agents (e.g., a pharmaceutically acceptable carrier, adjuvant, excipient, and the like). In some embodiments, a composition for stimulating an immune response of the present invention is administered in a manner to induce a humoral immune response. In some embodiments, a composition for stimulating an immune response of the present invention is administered in a manner to induce a cellular (e.g., cytotoxic T lymphocyte) immune response, rather than a humoral response. In some embodiments, a composition comprising a NE and an immunogen of the present invention induces both a cellular and humoral immune response.

The present invention is not limited by the type of NE utilized (e.g., in an immunogenic composition comprising an immunogen. Indeed, a variety of NE compositions are contemplated to be useful in the present invention.

For example, in some embodiments, a nanoemulsion (e.g., for inactivation of an orthopox virus (e.g., VV)) comprises (i) an aqueous phase; (ii) an oil phase; and at least one additional compound. In some embodiments of the present invention, these additional compounds are admixed into either the aqueous or oil phases of the composition. In other embodiments, these additional compounds are admixed into a composition of previously emulsified oil and aqueous phases. In certain of these embodiments, one or more additional compounds are admixed into an existing emulsion composition immediately prior to its use. In other embodiments, one or more additional compounds are admixed into an existing emulsion composition prior to the compositions immediate use.

Additional compounds suitable for use in a nanoemulsion of the present invention include, but are not limited to, one or more organic, and more particularly, organic phosphate based solvents, surfactants and detergents, cationic halogen containing compounds, germination enhancers, interaction enhancers, food additives (e.g., flavorings, sweetners, bulking agents, and the like) and pharmaceutically acceptable compounds. Certain exemplary embodiments of the various compounds contemplated for use in the compositions of the present invention are presented below.

A. Aqueous Phase

In certain preferred embodiments, a nanoemulsion comprises about 5 to 60, preferably 10 to 40, more preferably 15 to 30, vol. % aqueous phase, based on the total volume of the emulsion, although higher and lower amounts are contemplated. In preferred embodiments, the aqueous phase comprises water at a pH of about 4 to 10, preferably about 6 to 8. When the emulsions of the present invention contain a germination enhancer, the pH is preferably 6 to 8. The water is preferably deionized (hereinafter “DiH₂O”) or distilled. In some embodiments the aqueous phase comprises phosphate buffered saline (PBS). In those embodiments of the present invention intended for administration to, or contact with, a subject (e.g., a subject vaccinated with a composition of the present invention), the aqueous phase, and any additional compounds provided in the aqueous phase, may further be sterile and pyrogen free.

B. Oil Phase and Solvents

In certain preferred embodiments, the oil phase (e.g., carrier oil) of a nanoemulsion comprises 30-90, preferably 60-80, and more preferably 60-70, vol. % of oil, based on the total volume of the emulsion, although higher and lower amounts are contemplated. Suitable oils include, but are not limited to, soybean oil, avocado oil, flaxseed oil, coconut oil, cottonseed oil, squalene oil, olive oil, canola oil, corn oil, rapeseed oil, safflower oil, sunflower oil, pine oil (e.g., 15%), Olestra oil, fish oils, flavor oils, water insoluble vitamins and mixtures thereof. In particularly preferred embodiments, soybean oil is used. Additional contemplated oils include motor oils, mineral oils, and butter. In preferred embodiments of the present invention, the oil phase is preferably distributed throughout the aqueous phase as droplets having a mean particle size in the range from about 1-2 microns, more preferably from 0.2 to 0.8, and most preferably about 0.8 microns. In other embodiments, the aqueous phase can be distributed in the oil phase. In some preferred embodiments, very small droplet sizes are utilized (e.g., less than 0.5 microns) to produce stable nanoemulsion compositions. It is contemplated that small droplet compositions also provide clear solutions, which may find desired use in certain product types.

In some embodiments, the oil phase comprises 3-15, preferably 5-10 vol. % of an organic solvent, based on the total volume of the emulsion, although higher and lower amounts are contemplated. Although an understanding of the mechanism is not necessary to practice the present invention and the present invention is not limited to any particular mechanism of action, it is contemplated that the organic phosphate-based solvents employed in an emulsion serves to disrupt and inactivate the pathogen (e.g., disrupt lipids in membranes or viral envelopes). Thus, any solvent that can remove sterols or phospholipids finds use in the emulsions of the present invention. Suitable organic solvents include, but are not limited to, organic phosphate based solvents or alcohols. In preferred embodiments, the organic phosphate based solvents include, but are not limited to, dialkyl- and trialkyl phosphates (e.g., tri-n-butyl phosphate (TBP)) in any combination. A particularly preferred trialkyl phosphate in certain embodiments comprises tri-n-butyl phosphate, which is a plasticizer. Moreover, in a preferred embodiment, each alkyl group of the di- or trialkyl phosphate has from one to ten or more carbon atoms, more preferably two to eight carbon atoms. The present invention also contemplates that each alkyl group of the di- or trialkyl phosphate may or may not be identical to one another. In certain embodiments, mixtures of different dialkyl and trialkyl phosphates can be employed. In those embodiments comprising one or more alcohols as solvents, such solvents include, but are not limited to, methanol, ethanol, propanol and octanol. In a particularly preferred embodiment, the alcohol is ethanol. In those embodiments of the present invention intended for consumption by, or contact to, a host, the oil phase, and any additional compounds provided in the oil phase, may further be sterile and pyrogen free.

C. Surfactants and Detergents

In some embodiments, a nanoemulsion further comprises one or more surfactants or detergents (e.g., from about 3 to 15%, and preferably about 10%, although higher and lower amounts are contemplated). While the present invention is not limited to any particular mechanism, and an understanding of the mechanism is not required to practice the present invention, it is contemplated that surfactants help to stabilize the compositions (e.g., used to generate an immune response in a subject (e.g., used as a vaccine). Both non-ionic (non-anionic) and ionic surfactants are contemplated. Additionally, surfactants from the BRIJ family of surfactants find use in the compositions of the present invention. The surfactant can be provided in either the aqueous or the oil phase. Surfactants suitable for use with the emulsions include a variety of anionic and nonionic surfactants, as well as other emulsifying compounds that are capable of promoting the formation of oil-in-water emulsions. In general, emulsifying compounds are relatively hydrophilic, and blends of emulsifying compounds can be used to achieve the necessary qualities. In some formulations, nonionic surfactants have advantages over ionic emulsifiers in that they are substantially more compatible with a broad pH range and often form more stable emulsions than do ionic (e.g., soap-type) emulsifiers. Thus, in certain preferred embodiments, a nanoemulsion comprises one or more non-ionic surfactants such as a polysorbate surfactants (e.g., polyoxyethylene ethers), polysorbate detergents, pheoxypolyethoxyethanols, and the like. Examples of polysorbate detergents useful in the present invention include, but are not limited to, TWEEN 20, TWEEN 40, TWEEN 60, TWEEN 80, etc.

TWEEN 60 (polyoxyethylenesorbitan monostearate), together with TWEEN 20, TWEEN 40 and TWEEN 80, comprise polysorbates that are used as emulsifiers in a number of pharmaceutical compositions. In some embodiments of the present invention, these compounds are also used as co-components with adjuvants. TWEEN surfactants also appear to have virucidal effects on lipid-enveloped viruses (See e.g., Eriksson et al., Blood Coagulation and Fibtinolysis 5 (Suppl. 3):S37-S44 (1994)).

Examples of pheoxypolyethoxyethanols, and polymers thereof, useful in the present invention include, but are not limited to, TRITON (e.g., X-100, X-301, X-165, X-102, X-200), and TYLOXAPOL. TRITON X-100 is a strong non-ionic detergent and dispersing agent widely used to extract lipids and proteins from biological structures. It also has virucidal effect against broad spectrum of enveloped viruses (See e.g., Maha and Igarashi, Southeast Asian J. Trop. Med. Pub. Health 28:718 (1997); and Portocala et al., Virologie 27:261 (1976)). Due to this anti-viral activity, it is employed to inactivate viral pathogens in fresh frozen human plasma (See e.g., Horowitz et al., Blood 79:826 (1992)).

In particularly preferred embodiments, the surfactants TRITON X-100 (t-octylphenoxypolyethoxyethanol), and/or TYLOXAPOL are employed. Some other embodiments, employ spermicides (e.g., Nonoxynol-9). Additional surfactants and detergents useful in the compositions of the present invention may be ascertained from reference works (See e.g., McCutheon's Volume 1: Emulsions and Detergents—North American Edition, 2000).

D. Cationic Halogen Containing Compounds

In some embodiments, nanoemulsions (e.g., used in an immunogenic composition of the present invention) further comprise a cationic halogen containing compound (e.g., from about 0.5 to 1.0 wt. % or more, based on the total weight of the emulsion, although higher and lower amounts are contemplated). In preferred embodiments, the cationic halogen-containing compound is preferably premixed with the oil phase; however, it should be understood that the cationic halogen-containing compound may be provided in combination with the emulsion composition in a distinct formulation. Suitable halogen containing compounds may be selected, for example, from compounds comprising chloride, fluoride, bromide and iodide ions. In preferred embodiments, suitable cationic halogen containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

E. Germination Enhancers

In other embodiments of the present invention, nanoemulsion compositions further comprise one or more germination enhancing compounds (e.g., from about 1 mM to 15 mM, and more preferably from about 5 mM to 10 mM, although higher and lower amounts are contemplated). In preferred embodiments, the germination enhancing compound is provided in the aqueous phase prior to formation of the emulsion. The present invention contemplates that when germination enhancers are added to the disclosed compositions the sporicidal properties of the compositions are enhanced. The present invention further contemplates that such germination enhancers initiate sporicidal activity near neutral pH (between pH 6-8, and preferably 7). Such neutral pH emulsions can be obtained, for example, by diluting with phosphate buffer saline (PBS) or by preparations of neutral emulsions. The sporicidal activity of the compositions preferentially occurs when the spores initiate germination.

In certain embodiments, suitable germination enhancing agents of the invention include, but are not limited to, α-amino acids comprising glycine and the L-enantiomers of alanine, valine, leucine, isoleucine, serine, threonine, lysine, phenylalanine, tyrosine, and the alkyl esters thereof. Additional information on the effects of amino acids on germination may be found in U.S. Pat. No. 5,510,104, herein incorporated by reference in its entirety. In some embodiments, a mixture of glucose, fructose, asparagine, sodium chloride (NaCl), ammonium chloride (NH₄Cl), calcium chloride (CaCl₂) and potassium chloride (KCl) also may be used. In particularly preferred embodiments of the present invention, the formulation comprises the germination enhancers L-alanine, CaCl₂, Inosine and NH₄Cl. In some embodiments, the compositions further comprise one or more common forms of growth media (e.g., trypticase soy broth, and the like) that additionally may or may not itself comprise germination enhancers and buffers.

The above compounds are merely exemplary germination enhancers and it is understood that other known germination enhancers will find use in the compositions of the present invention. A candidate germination enhancer should meet two criteria for inclusion in the compositions of the present invention: it should be capable of being associated with a nanoemulsion and it should increase the rate of germination of a target spore when incorporated in the emulsions of the present invention.

F. Interaction Enhancers

In still other embodiments, a nanoemulsion may comprise one or more compounds capable of increasing the interaction of the nanoemulsion (i.e., “interaction enhancer”) with a target pathogen (e.g., with a viral envelope of an orthopox virus neutralized with the nanoemulsion). In some embodiments, the interaction enhancer is premixed with the oil phase; however, in other embodiments the interaction enhancer is provided in combination with the compositions after emulsification. In certain preferred embodiments, the interaction enhancer is a chelating agent (e.g., ethylenediaminetetraacetic acid (EDTA) or ethylenebis(oxyethylenenitrilo)tetraacetic acid (EGTA) in a buffer (e.g., tris buffer)). It is understood that chelating agents are merely exemplary interaction enhancing compounds. Indeed, other agents that increase the interaction of a nanoemulsion with a pathogen (e.g., an orthopox virus (e.g., VV)) are contemplated. In particularly preferred embodiments, the interaction enhancer is at a concentration of about 50 to about 250 μM, although higher and lower amounts are contemplated. One skilled in the art will be able to determine whether a particular agent has the desired function of acting as an interaction enhancer by applying such an agent in combination with a nanoemulsion composition of the present invention to a target pathogen (e.g., orthopox virus) and comparing the inactivation of the target when contacted by the admixture with inactivation of like targets by the composition of the present invention without the agent. For example, an agent that increases the interaction and thereby neutralizes (e.g., decreases or inhibits the growth of the pathogen) in comparison to that parameter in its absence is considered an interaction enhancer.

G. Other Components

In some embodiments, a nanoemulsion comprises one or more additional components that provide a desired property or functionality to the nanoemulsions. These components may be incorporated into the aqueous phase or the oil phase of the nanoemulsions and/or may be added prior to or following emulsification. For example, in some embodiments, the nanoemulsions further comprise phenols (e.g., triclosan, phenyl phenol), acidifying agents (e.g., citric acid (e.g., 1.5-6%), acetic acid, lemon juice), alkylating agents (e.g., sodium hydroxide (e.g., 0.3%)), buffers (e.g., citrate buffer, acetate buffer, and other buffers useful to maintain a specific pH), and halogens (e.g., polyvinylpyrrolidone, sodium hypochlorite, hydrogen peroxide).

Exemplary techniques for making a nanoemulsion (e.g., used to inactivate an orthopox virus (e.g., vaccinia virus) are described below. Additionally, a number of specific, although exemplary, formulation recipes are also set forth below.

Formulation Techniques

Nanoemulsions of the present invention can be formed using classic emulsion forming techniques. In brief, the oil phase is mixed with the aqueous phase under relatively high shear forces (e.g., using high hydraulic and mechanical forces) to obtain an oil-in-water nanoemulsion. The emulsion is formed by blending the oil phase with an aqueous phase on a volume-to-volume basis ranging from about 1:9 to 5:1, preferably about 5:1 to 3:1, most preferably 4:1, oil phase to aqueous phase. The oil and aqueous phases can be blended using any apparatus capable of producing shear forces sufficient to form an emulsion such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In preferred embodiments, compositions used in the methods of the present invention comprise droplets of an oily discontinuous phase dispersed in an aqueous continuous phase, such as water. In preferred embodiments, nanoemulsions of the present invention are stable, and do not decompose even after long storage periods (e.g., greater than one or more years). Furthermore, in some embodiments, nanoemulsions are stable (e.g., in some embodiments for greater than 3 months, in some embodiments for greater than 6 months, in some embodiments for greater than 12 months, in some embodiments for greater than 18 months) after combination with an immunogen (e.g., a pathogen (e.g., orthopox virus (e.g., vaccinia virus))). In preferred embodiments, nanoemulsions of the present invention are non-toxic and safe when administered (e.g., via spraying or contacting mucosal surfaces, swallowed, inhaled, etc.) to a subject.

In some embodiments, a portion of the emulsion may be in the form of lipid structures including, but not limited to, unilamellar, multilamellar, and paucliamellar lipid vesicles, micelles, and lamellar phases.

Some embodiments of the present invention employ an oil phase containing ethanol. For example, in some embodiments, the emulsions of the present invention contain (i) an aqueous phase and (ii) an oil phase containing ethanol as the organic solvent and optionally a germination enhancer, and (iii) TYLOXAPOL as the surfactant (preferably 2-5%, more preferably 3%). This formulation is highly efficacious for inactivation of pathogens and is also non-irritating and non-toxic to mammalian subjects (e.g., and thus can be used for administration to a mucosal surface).

In some other embodiments, the emulsions of the present invention comprise a first emulsion emulsified within a second emulsion, wherein (a) the first emulsion comprises (i) an aqueous phase; and (ii) an oil phase comprising an oil and an organic solvent; and (iii) a surfactant; and (b) the second emulsion comprises (i) an aqueous phase; and (ii) an oil phase comprising an oil and a cationic containing compound; and (iii) a surfactant.

Exemplary Formulations

The following description provides a number of exemplary emulsions including formulations for compositions BCTP and X₈W₆₀PC. BCTP comprises a water-in oil nanoemulsion, in which the oil phase was made from soybean oil, tri-n-butyl phosphate, and TRITON X-100 in 80% water. X₈W₆₀PC comprises a mixture of equal volumes of BCTP with W₈₀8P. W₈₀8P is a liposome-like compound made of glycerol monostearate, refined oya sterols (e.g., GENEROL sterols), TWEEN 60, soybean oil, a cationic ion halogen-containing CPC and peppermint oil. The GENEROL family are a group of a polyethoxylated soya sterols (Henkel Corporation, Ambler, Pa.). Exemplary emulsion formulations useful in the present invention are provided in Table 1. These particular formulations may be found in U.S. Pat. No. 5,700,679 (NN); U.S. Pat. Nos. 5,618,840; 5,549,901 (W₈₀8P); and U.S. Pat. No. 5,547,677, each of which is hereby incorporated by reference in their entireties. Certain other emulsion formulations are presented U.S. patent application Ser. No. 10/669,865, hereby incorporated by reference in its entirety.

The X₈W₆₀PC emulsion is manufactured by first making the W₈₀8P emulsion and BCTP emulsions separately. A mixture of these two emulsions is then re-emulsified to produce a fresh emulsion composition termed X₈W₆₀PC. Methods of producing such emulsions are described in U.S. Pat. Nos. 5,103,497 and 4,895,452 (each of which is herein incorporated by reference in their entireties).

TABLE 1

| | | | Water to Oil |
|---|---|---|---|
| | Oil Phase Formula | | Phase Ratio (Vol/Vol) |
| BCTP | 1 | vol. Tri(N-butyl)phosphate | 4:1 |
| | 1 | vol. TRITON X-100 | |
| | 8 | vol. Soybean oil | |
| NN | 86.5 | g Glycerol monooleate | 3:1 |
| | 60.1 | ml Nonoxynol-9 | |
| | 24.2 | g GENEROL 122 | |
| | 3.27 | g Cetylpyridinium chloride | |
| | 554 | g Soybean oil | |
| W₈₀8P | 86.5 | g Glycerol monooleate | 3.2:1 |
| | 21.2 | g Polysorbate 60 | |
| | 24.2 | g GENEROL 122 | |
| | 3.27 | g Cetylpyddinium chloride | |
| | 4 | ml Peppermint oil | |
| | 554 | g Soybean oil | |
| SS | 86.5 | g Glycerol monooleate | 3.2:1 |
| | 21.2 | g Polysorbate 60 | (1% bismuth in water) |
| | 24.2 | g GENEROL 122 | |
| | 3.27 | g Cetylpyridinium chloride | |
| | 554 | g Soybean oil | |

The compositions listed above are only exemplary and those of skill in the art will be able to alter the amounts of the components to arrive at a nanoemulsion composition suitable for the purposes of the present invention. Those skilled in the art will understand that the ratio of oil phase to water as well as the individual oil carrier, surfactant CPC and organic phosphate buffer, components of each composition may vary.

Although certain compositions comprising BCTP have a water to oil ratio of 4:1, it is understood that the BCTP may be formulated to have more or less of a water phase. For example, in some embodiments, there is 3, 4, 5, 6, 7, 8, 9, 10, or more parts of the water phase to each part of the oil phase. The same holds true for the W₈₀8P formulation. Similarly, the ratio of Tri(N-butyl)phosphate:TRITON X-100:soybean oil also may be varied.

Although Table 1 lists specific amounts of glycerol monooleate, polysorbate 60, GENEROL 122, cetylpyridinium chloride, and carrier oil for W₈₀8P, these are merely exemplary. An emulsion that has the properties of W₈₀8P may be formulated that has different concentrations of each of these components or indeed different components that will fulfill the same function. For example, the emulsion may have between about 80 to about 100 g of glycerol monooleate in the initial oil phase. In other embodiments, the emulsion may have between about 15 to about 30 g polysorbate 60 in the initial oil phase. In yet another embodiment the composition may comprise between about 20 to about 30 g of a GENEROL sterol, in the initial oil phase.

Individual components of nanoemulsions (e.g. in an immunogenic composition of the present invention) can function both to inactivate a pathogen (e.g., orthopox virus (e.g., vaccinia virus)) as well as to contribute to the non-toxicity of the emulsions. For example, the active component in BCTP, TRITON-X100, shows less ability to inactivate a virus at concentrations equivalent to 11% BCTP. Adding the oil phase to the detergent and solvent markedly reduces the toxicity of these agents in tissue culture at the same concentrations. While not being bound to any theory (an understanding of the mechanism is not necessary to practice the present invention, and the present invention is not limited to any particular mechanism), it is suggested that the nanoemulsion enhances the interaction of its components with the pathogens thereby facilitating the inactivation of the pathogen and reducing the toxicity of the individual components. Furthermore, when all the components of BCTP are combined in one composition but are not in a nanoemulsion structure, the mixture is not as effective at inactivating a pathogen (e.g., orthopox virus (e.g., vaccinia virus)) as when the components are in a nanoemulsion structure.

Numerous additional embodiments presented in classes of formulations with like compositions are presented below. The following compositions recite various ratios and mixtures of active components. One skilled in the art will appreciate that the below recited formulation are exemplary and that additional formulations comprising similar percent ranges of the recited components are within the scope of the present invention.

In certain embodiments of the present invention, a nanoemulsion comprises from about 3 to 8 vol. % of TYLOXAPOL, about 8 vol. % of ethanol, about 1 vol. % of cetylpyridinium chloride (CPC), about 60 to 70 vol. % oil (e.g., soybean oil), about 15 to 25 vol. % of aqueous phase (e.g., DiH₂O or PBS), and in some formulations less than about 1 vol. % of 1N NaOH. Some of these embodiments comprise PBS. It is contemplated that the addition of 1N NaOH and/or PBS in some of these embodiments, allows the user to advantageously control the pH of the formulations, such that pH ranges from about 7.0 to about 9.0, and more preferably from about 7.1 to 8.5 are achieved. For example, one embodiment of the present invention comprises about 3 vol. % of TYLOXAPOL, about 8 vol. % of ethanol, about 1 vol. % of CPC, about 64 vol. % of soybean oil, and about 24 vol. % of DiH₂O (designated herein as Y3EC). Another similar embodiment comprises about 3.5 vol. % of TYLOXAPOL, about 8 vol. % of ethanol, and about 1 vol. % of CPC, about 64 vol. % of soybean oil, and about 23.5 vol. % of DiH₂O (designated herein as Y3.5EC). Yet another embodiment comprises about 3 vol. % of TYLOXAPOL, about 8 vol. % of ethanol, about 1 vol. % of CPC, about 0.067 vol. % of 1N NaOH, such that the pH of the formulation is about 7.1, about 64 vol. % of soybean oil, and about 23.93 vol. % of DiH₂O (designated herein as Y3EC pH 7.1). Still another embodiment comprises about 3 vol. % of TYLOXAPOL, about 8 vol. % of ethanol, about 1 vol. % of CPC, about 0.67 vol. % of 1N NaOH, such that the pH of the formulation is about 8.5, and about 64 vol. % of soybean oil, and about 23.33 vol. % of DiH₂O (designated herein as Y3EC pH 8.5). Another similar embodiment comprises about 4% TYLOXAPOL, about 8 vol. % ethanol, about 1% CPC, and about 64 vol. % of soybean oil, and about 23 vol. % of DiH₂O (designated herein as Y4EC). In still another embodiment the formulation comprises about 8% TYLOXAPOL, about 8% ethanol, about 1 vol. % of CPC, and about 64 vol. % of soybean oil, and about 19 vol. % of DiH₂O (designated herein as Y8EC). A further embodiment comprises about 8 vol. % of TYLOXAPOL, about 8 vol. % of ethanol, about 1 vol. % of CPC, about 64 vol. % of soybean oil, and about 19 vol. % of 1× PBS (designated herein as Y8EC PBS).

In some embodiments of the present invention, a nanoemulsion comprises about 8 vol. % of ethanol, and about 1 vol. % of CPC, and about 64 vol. % of oil (e.g., soybean oil), and about 27 vol. % of aqueous phase (e.g., DiH₂O or PBS) (designated herein as EC).

In some embodiments, a nanoemulsion comprises from about 8 vol. % of sodium dodecyl sulfate (SDS), about 8 vol. % of tributyl phosphate (TBP), and about 64 vol. % of oil (e.g., soybean oil), and about 20 vol. % of aqueous phase (e.g., DiH₂O or PBS) (designated herein as S8P).

In some embodiments, a nanoemulsion comprises from about 1 to 2 vol. % of TRITON X-100, from about 1 to 2 vol. % of TYLOXAPOL, from about 7 to 8 vol. % of ethanol, about 1 vol. % of cetylpyridinium chloride (CPC), about 64 to 57.6 vol. % of oil (e.g., soybean oil), and about 23 vol. % of aqueous phase (e.g., DiH₂O or PBS). Additionally, some of these formulations further comprise about 5 mM of L-alanine/Inosine, and about 10 mM ammonium chloride. Some of these formulations comprise PBS. It is contemplated that the addition of PBS in some of these embodiments, allows the user to advantageously control the pH of the formulations. For example, one embodiment of the present invention comprises about 2 vol. % of TRITON X-100, about 2 vol. % of TYLOXAPOL, about 8 vol. % of ethanol, about 1 vol. % CPC, about 64 vol. % of soybean oil, and about 23 vol. % of aqueous phase DiH₂O. In another embodiment the formulation comprises about 1.8 vol. % of TRITON X-100, about 1.8 vol. % of TYLOXAPOL, about 7.2 vol. % of ethanol, about 0.9 vol. % of CPC, about 5 mM L-alanine/Inosine, and about 10 mM ammonium chloride, about 57.6 vol. % of soybean oil, and the remainder of 1× PBS (designated herein as 90% X2Y2EC/GE).

In alternative embodiments, a nanoemulsion comprises from about 5 vol. % of TWEEN 80, from about 8 vol. % of ethanol, from about 1 vol. % of CPC, about 64 vol. % of oil (e.g., soybean oil), and about 22 vol. % of DiH₂O (designated herein as W₈₀5EC).

In still other embodiments of the present invention, a nanoemulsion comprises from about 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC).

In still other embodiments of the present invention, a nanoemulsion comprises from about 2 to 8 vol. % of TRITON X-100, about 8 vol. % of ethanol, about 1 vol. % of CPC, about 60 to 70 vol. % of oil (e.g., soybean, or olive oil), and about 15 to 25 vol. % of aqueous phase (e.g., DiH₂O or PBS). For example, the present invention contemplates formulations comprising about 2 vol. % of TRITON X-100, about 8 vol. % of ethanol, about 64 vol. % of soybean oil, and about 26 vol. % of DiH₂O (designated herein as X2E). In other similar embodiments, a nanoemulsion comprises about 3 vol. % of TRITON X-100, about 8 vol. % of ethanol, about 64 vol. % of soybean oil, and about 25 vol. % of DiH₂O (designated herein as X3E). In still further embodiments, the formulations comprise about 4 vol. % Triton of X-100, about 8 vol. % of ethanol, about 64 vol. % of soybean oil, and about 24 vol. % of DiH₂O (designated herein as X4E). In yet other embodiments, a nanoemulsion comprises about 5 vol. % of TRITON X-100, about 8 vol. % of ethanol, about 64 vol. % of soybean oil, and about 23 vol. % of DiH₂O (designated herein as X5E). In some embodiments, a nanoemulsion comprises about 6 vol. % of TRITON X-100, about 8 vol. % of ethanol, about 64 vol. % of soybean oil, and about 22 vol. % of DiH₂O (designated herein as X6E). In still further embodiments of the present invention, a nanoemulsion comprises about 8 vol. % of TRITON X-100, about 8 vol. % of ethanol, about 64 vol. % of soybean oil, and about 20 vol. % of DiH₂O (designated herein as X8E). In still further embodiments, a nanoemulsion comprises about 8 vol. % of TRITON X-100, about 8 vol. % of ethanol, about 64 vol. % of olive oil, and about 20 vol. % of DiH₂O (designated herein as X8E O). In yet another embodiment, a nanoemulsion comprises 8 vol. % of TRITON X-100, about 8 vol. % ethanol, about 1 vol. % CPC, about 64 vol. % of soybean oil, and about 19 vol. % of DiH₂O (designated herein as X8EC).

In alternative embodiments of the present invention, a nanoemulsion comprises from about 1 to 2 vol. % of TRITON X-100, from about 1 to 2 vol. % of TYLOXAPOL, from about 6 to 8 vol. % TBP, from about 0.5 to 1.0 vol. % of CPC, from about 60 to 70 vol. % of oil (e.g., soybean), and about 1 to 35 vol. % of aqueous phase (e.g., DiH₂O or PBS). Additionally, certain of these nanoemulsions may comprise from about 1 to 5 vol. % of trypticase soy broth, from about 0.5 to 1.5 vol. % of yeast extract, about 5 mM L-alanine/Inosine, about 10 mM ammonium chloride, and from about 20-40 vol. % of liquid baby formula. In some embodiments comprising liquid baby formula, the formula comprises a casein hydrolysate (e.g., Neutramigen, or Progestimil, and the like). In some of these embodiments, a nanoemulsion further comprises from about 0.1 to 1.0 vol. % of sodium thiosulfate, and from about 0.1 to 1.0 vol. % of sodium citrate. Other similar embodiments comprising these basic components employ phosphate buffered saline (PBS) as the aqueous phase. For example, one embodiment comprises about 2 vol. % of TRITON X-100, about 2 vol. % TYLOXAPOL, about 8 vol. % TBP, about 1 vol. % of CPC, about 64 vol. % of soybean oil, and about 23 vol. % of DiH₂O (designated herein as X2Y2EC). In still other embodiments, the inventive formulation comprises about 2 vol. % of TRITON X-100, about 2 vol. % TYLOXAPOL, about 8 vol. % TBP, about 1 vol. % of CPC, about 0.9 vol. % of sodium thiosulfate, about 0.1 vol. % of sodium citrate, about 64 vol. % of soybean oil, and about 22 vol. % of DiH₂O (designated herein as X2Y2PC STS1). In another similar embodiment, a nanoemulsion comprises about 1.7 vol. % TRITON X-100, about 1.7 vol. % TYLOXAPOL, about 6.8 vol. % TBP, about 0.85% CPC, about 29.2% NEUTRAMIGEN, about 54.4 vol. % of soybean oil, and about 4.9 vol. % of DiH₂O (designated herein as 85% X2Y2PC/baby). In yet another embodiment of the present invention, a nanoemulsion comprises about 1.8 vol. % of TRITON X-100, about 1.8 vol. % of TYLOXAPOL, about 7.2 vol. % of TBP, about 0.9 vol. % of CPC, about 5 mM L-alanine/Inosine, about 10 mM ammonium chloride, about 57.6 vol. % of soybean oil, and the remainder vol. % of 0.1× PBS (designated herein as 90% X2Y2 PC/GE). In still another embodiment, a nanoemulsion comprises about 1.8 vol. % of TRITON X-100, about 1.8 vol. % of TYLOXAPOL, about 7.2 vol. % TBP, about 0.9 vol. % of CPC, and about 3 vol. % trypticase soy broth, about 57.6 vol. % of soybean oil, and about 27.7 vol. % of DiH₂O (designated herein as 90% X2Y2PC/TSB). In another embodiment of the present invention, a nanoemulsion comprises about 1.8 vol. % TRITON X-100, about 1.8 vol. % TYLOXAPOL, about 7.2 vol. % TBP, about 0.9 vol. % CPC, about 1 vol. % yeast extract, about 57.6 vol. % of soybean oil, and about 29.7 vol. % of DiH₂O (designated herein as 90% X2Y2PC/YE).

In some embodiments of the present invention, a nanoemulsion comprises about 3 vol. % of TYLOXAPOL, about 8 vol. % of TBP, and about 1 vol. % of CPC, about 60 to 70 vol. % of oil (e.g., soybean or olive oil), and about 15 to 30 vol. % of aqueous phase (e.g., DiH₂O or PBS). In a particular embodiment of the present invention, a nanoemulsion comprises about 3 vol. % of TYLOXAPOL, about 8 vol. % of TBP, and about 1 vol. % of CPC, about 64 vol. % of soybean, and about 24 vol. % of DiH₂O (designated herein as Y3PC).

In some embodiments of the present invention, a nanoemulsion comprises from about 4 to 8 vol. % of TRITON X-100, from about 5 to 8 vol. % of TBP, about 30 to 70 vol. % of oil (e.g., soybean or olive oil), and about 0 to 30 vol. % of aqueous phase (e.g., DiH₂0 or PBS). Additionally, certain of these embodiments further comprise about 1 vol. % of CPC, about 1 vol. % of benzalkonium chloride, about 1 vol. % cetylyridinium bromide, about 1 vol. % cetyldimethyletylammonium bromide, 500 μM EDTA, about 10 mM ammonium chloride, about 5 mM Inosine, and about 5 mM L-alanine. For example, in a certain preferred embodiment, a nanoemulsion comprises about 8 vol. % of TRITON X-100, about 8 vol. % of TBP, about 64 vol. % of soybean oil, and about 20 vol. % of DiH₂O (designated herein as X8P). In another embodiment of the present invention, a nanoemulsion comprises about 8 vol. % of TRITON X-100, about 8 vol. % of TBP, about 1% of CPC, about 64 vol. % of soybean oil, and about 19 vol. % of DiH₂O (designated herein as X8PC). In still another embodiment, a nanoemulsion comprises about 8 vol. % TRITON X-100, about 8 vol. % of TBP, about 1 vol. % of CPC, about 50 vol. % of soybean oil, and about 33 vol. % of DiH₂O (designated herein as ATB-X1001). In yet another embodiment, the formulations comprise about 8 vol. % of TRITON X-100, about 8 vol. % of TBP, about 2 vol. % of CPC, about 50 vol. % of soybean oil, and about 32 vol. % of DiH₂O (designated herein as ATB-X002). In some embodiments, a nanoemulsion comprises about 4 vol. % TRITON X-100, about 4 vol. % of TBP, about 0.5 vol. % of CPC, about 32 vol. % of soybean oil, and about 59.5 vol. % of DiH₂O (designated herein as 50% X8PC). In some embodiments, a nanoemulsion comprises about 8 vol. % of TRITON X-100, about 8 vol. % of TBP, about 0.5 vol. % CPC, about 64 vol. % of soybean oil, and about 19.5 vol. % of DiH₂O (designated herein as X8PC_{1/2}). In some embodiments of the present invention, a nanoemulsion comprises about 8 vol. % of TRITON X-100, about 8 vol. % of TBP, about 2 vol. % of CPC, about 64 vol. % of soybean oil, and about 18 vol. % of DiH₂O (designated herein as X8PC2). In other embodiments, a nanoemulsion comprises about 8 vol. % of TRITON X-100, about 8% of TBP, about 1% of benzalkonium chloride, about 50 vol. % of soybean oil, and about 33 vol. % of DiH₂O (designated herein as X8P BC). In an alternative embodiment of the present invention, a nanoemulsion comprises about 8 vol. % of TRITON X-100, about 8 vol. % of TBP, about 1 vol. % of cetylyridinium bromide, about 50 vol. % of soybean oil, and about 33 vol. % of DiH₂O (designated herein as X8P CPB). In another exemplary embodiment of the present invention, a nanoemulsion comprises about 8 vol. % of TRITON X-100, about 8 vol. % of TBP, about 1 vol. % of cetyldimethyletylammonium bromide, about 50 vol. % of soybean oil, and about 33 vol. % of DiH₂O (designated herein as X8P CTAB). In still further embodiments, a nanoemulsion comprises about 8 vol. % of TRITON X-100, about 8 vol. % of TBP, about 1 vol. % of CPC, about 500 μM EDTA, about 64 vol. % of soybean oil, and about 15.8 vol. % DiH₂0 (designated herein as X8PC EDTA). In some embodiments, a nanoemulsion comprises 8 vol. % of TRITON X-100, about 8 vol. % of TBP, about 1 vol. % of CPC, about 10 mM ammonium chloride, about 5 mM Inosine, about 5 mM L-alanine, about 64 vol. % of soybean oil, and about 19 vol. % of DiH₂O or PBS (designated herein as X8PC GE_{1×}). In another embodiment of the present invention, a nanoemulsion comprises about 5 vol. % of TRITON X-100, about 5% of TBP, about 1 vol. % of CPC, about 40 vol. % of soybean oil, and about 49 vol. % of DiH₂O (designated herein as X5P₅C).

In some embodiments of the present invention, a nanoemulsion comprises about 2 vol. % TRITON X-100, about 6 vol. % TYLOXAPOL, about 8 vol. % ethanol, about 64 vol. % of soybean oil, and about 20 vol. % of DiH₂O (designated herein as X2Y6E).

In an additional embodiment of the present invention, a nanoemulsion comprises about 8 vol. % of TRITON X-100, and about 8 vol. % of glycerol, about 60 to 70 vol. % of oil (e.g., soybean or olive oil), and about 15 to 25 vol. % of aqueous phase (e.g., DiH₂O or PBS). Certain nanoemulsion compositions (e.g., used to generate an immune response (e.g., for use as a vaccine) comprise about 1 vol. % L-ascorbic acid. For example, one particular embodiment comprises about 8 vol. % of TRITON X-100, about 8 vol. % of glycerol, about 64 vol. % of soybean oil, and about 20 vol. % of DiH₂O (designated herein as X8G). In still another embodiment, a nanoemulsion comprises about 8 vol. % of TRITON X-100, about 8 vol. % of glycerol, about 1 vol. % of L-ascorbic acid, about 64 vol. % of soybean oil, and about 19 vol. % of DiH₂O (designated herein as X8GV_{c}).

In still further embodiments, a nanoemulsion comprises about 8 vol. % of TRITON X-100, from about 0.5 to 0.8 vol. % of TWEEN 60, from about 0.5 to 2.0 vol. % of CPC, about 8 vol. % of TBP, about 60 to 70 vol. % of oil (e.g., soybean or olive oil), and about 15 to 25 vol. % of aqueous phase (e.g., DiH₂O or PBS). For example, in one particular embodiment a nanoemulsion comprises about 8 vol. % of TRITON X-100, about 0.70 vol. % of TWEEN 60, about 1 vol. % of CPC, about 8 vol. % of TBP, about 64 vol. % of soybean oil, and about 18.3 vol. % of DiH₂O (designated herein as X8W60PC₁). In some embodiments, a nanoemulsion comprises about 8 vol. % of TRITON X-100, about 0.71 vol. % of TWEEN 60, about 1 vol. % of CPC, about 8 vol. % of TBP, about 64 vol. % of soybean oil, and about 18.29 vol. % of DiH₂O (designated herein as W60_{0.7}X8PC). In yet other embodiments, a nanoemulsion comprises from about 8 vol. % of TRITON X-100, about 0.7 vol. % of TWEEN 60, about 0.5 vol. % of CPC, about 8 vol. % of TBP, about 64 to 70 vol. % of soybean oil, and about 18.8 vol. % of DiH₂O (designated herein as X8W60PC₂). In still other embodiments, a nanoemulsion comprises about 8 vol. % of TRITON X-100, about 0.71 vol. % of TWEEN 60, about 2 vol. % of CPC, about 8 vol. % of TBP, about 64 vol. % of soybean oil, and about 17.3 vol. % of DiH₂O. In another embodiment of the present invention, a nanoemulsion comprises about 0.71 vol. % of TWEEN 60, about 1 vol. % of CPC, about 8 vol. % of TBP, about 64 vol. % of soybean oil, and about 25.29 vol. % of DiH₂O (designated herein as W60_{0.7}PC).

In another embodiment of the present invention, a nanoemulsion comprises about 2 vol. % of dioctyl sulfosuccinate, either about 8 vol. % of glycerol, or about 8 vol. % TBP, in addition to, about 60 to 70 vol. % of oil (e.g., soybean or olive oil), and about 20 to 30 vol. % of aqueous phase (e.g., DiH₂O or PBS). For example, in some embodiments, a nanoemulsion comprises about 2 vol. % of dioctyl sulfosuccinate, about 8 vol. % of glycerol, about 64 vol. % of soybean oil, and about 26 vol. % of DiH₂O (designated herein as D2G). In another related embodiment, a nanoemulsion comprises about 2 vol. % of dioctyl sulfosuccinate, and about 8 vol. % of TBP, about 64 vol. % of soybean oil, and about 26 vol. % of DiH₂O (designated herein as D2P).

In still other embodiments of the present invention, a nanoemulsion comprises about 8 to 10 vol. % of glycerol, and about 1 to 10 vol. % of CPC, about 50 to 70 vol. % of oil (e.g., soybean or olive oil), and about 15 to 30 vol. % of aqueous phase (e.g., DiH₂0 or PBS). Additionally, in certain of these embodiments, a nanoemulsion further comprises about 1 vol. % of L-ascorbic acid. For example, in some embodiments, a nanoemulsion comprises about 8 vol. % of glycerol, about 1 vol. % of CPC, about 64 vol. % of soybean oil, and about 27 vol. % of DiH₂O (designated herein as GC). In some embodiments, a nanoemulsion comprises about 10 vol. % of glycerol, about 10 vol. % of CPC, about 60 vol. % of soybean oil, and about 20 vol. % of DiH₂O (designated herein as GC10). In still another embodiment of the present invention, a nanoemulsion comprises about 10 vol. % of glycerol, about 1 vol. % of CPC, about 1 vol. % of L-ascorbic acid, about 64 vol. % of soybean or oil, and about 24 vol. % of DiH₂O (designated herein as GCV_{c}).

In some embodiments of the present invention, a nanoemulsion comprises about 8 to 10 vol. % of glycerol, about 8 to 10 vol. % of SDS, about 50 to 70 vol. % of oil (e.g., soybean or olive oil), and about 15 to 30 vol. % of aqueous phase (e.g., DiH₂O or PBS). Additionally, in certain of these embodiments, a nanoemulsion further comprise about 1 vol. % of lecithin, and about 1 vol. % of p-Hydroxybenzoic acid methyl ester. Exemplary embodiments of such formulations comprise about 8 vol. % SDS, 8 vol. % of glycerol, about 64 vol. % of soybean oil, and about 20 vol. % of DiH₂O (designated herein as S8G). A related formulation comprises about 8 vol. % of glycerol, about 8 vol. % of SDS, about 1 vol. % of lecithin, about 1 vol. % of p-Hydroxybenzoic acid methyl ester, about 64 vol. % of soybean oil, and about 18 vol. % of DiH₂O (designated herein as S8GL1B1).

In yet another embodiment of the present invention, a nanoemulsion comprises about 4 vol. % of TWEEN 80, about 4 vol. % of TYLOXAPOL, about 1 vol. % of CPC, about 8 vol. % of ethanol, about 64 vol. % of soybean oil, and about 19 vol. % of DiH₂O (designated herein as W₈₀4Y4EC).

In some embodiments of the present invention, a nanoemulsion comprises about 0.01 vol. % of CPC, about 0.08 vol. % of TYLOXAPOL, about 10 vol. % of ethanol, about 70 vol. % of soybean oil, and about 19.91 vol. % of DiH₂O (designated herein as Y.08EC.01).

In yet another embodiment of the present invention, a nanoemulsion comprises about 8 vol. % of sodium lauryl sulfate, and about 8 vol. % of glycerol, about 64 vol. % of soybean oil, and about 20 vol. % of DiH₂O (designated herein as SLS8G).

The specific formulations described above are simply examples to illustrate the variety of nanoemulsions that find use (e.g., to inactivate and/or neutralize orthopox virus (e.g., VV), and for generating an immune response in a subject (e.g., for use as a vaccine)) in the present invention. The present invention contemplates that many variations of the above formulations, as well as additional nanoemulsions, find use in the methods of the present invention. Candidate emulsions can be easily tested to determine if they are suitable. First, the desired ingredients are prepared using the methods described herein, to determine if an emulsion can be formed. If an emulsion cannot be formed, the candidate is rejected. For example, a candidate composition made of 4.5% sodium thiosulfate, 0.5% sodium citrate, 10% n-butanol, 64% soybean oil, and 21% DiH₂O does not form an emulsion.

Second, the candidate emulsion should form a stable emulsion. An emulsion is stable if it remains in emulsion form for a sufficient period to allow its intended use (e.g., to generate an immune response in a subject). For example, for emulsions that are to be stored, shipped, etc., it may be desired that the composition remain in emulsion form for months to years. Typical emulsions that are relatively unstable, will lose their form within a day. For example, a candidate composition made of 8% 1-butanol, 5% Tween 10, 1% CPC, 64% soybean oil, and 22% DiH₂O does not form a stable emulsion. Nanoemulsions that have been shown to be stable include, but are not limited to, 8 vol. % of TRITON X-100, about 8 vol. % of TBP, about 64 vol. % of soybean oil, and about 20 vol. % of DiH₂O (designated herein as X8P); 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC); 0.08% Triton X-100, 0.08% Glycerol, 0.01% Cetylpyridinium Chloride, 99% Butter, and 0.83% diH₂O (designated herein as 1% X8GC Butter); 0.8% Triton X-100, 0.8% Glycerol, 0.1% Cetylpyridinium Chloride, 6.4% Soybean Oil, 1.9% diH₂O, and 90% Butter (designated herein as 10% X8GC Butter); 2% W₂₀5EC, 1% Natrosol 250L NF, and 97% diH₂O (designated herein as 2% W₂₀5EC L GEL); 1% Cetylpyridinium Chloride, 5% Tween 20, 8% Ethanol, 64% 70 Viscosity Mineral Oil, and 22% diH₂O (designated herein as W₂₀5EC 70 Mineral Oil); 1% Cetylpyridinium Chloride, 5% Tween 20, 8% Ethanol, 64% 350 Viscosity Mineral Oil, and 22% diH₂O (designated herein as W₂₀5EC 350 Mineral Oil). In some embodiments, nanoemulsions of the present invention are stable for over a week, over a month, or over a year.

Third, the candidate emulsion should have efficacy for its intended use. For example, a nanoemuslion should inactivate (e.g., kill or inhibit growth of) virus (e.g., orthopox virus (e.g., VV)) to a desired level (e.g., 1 log, 2 log, 3 log, 4 log, . . . reduction). Using the methods described herein, one is capable of determining the suitability of a particular candidate emulsion against the desired pathogen. Generally, this involves exposing the pathogen to the emulsion for one or more time periods in a side-by-side experiment with the appropriate control samples (e.g., a negative control such as water) and determining if, and to what degree, the emulsion inactivates (e.g., kills and/or neutralizes) the microorganism. For example, a candidate composition made of 1% ammonium chloride, 5% Tween 20, 8% ethanol, 64% soybean oil, and 22% DiH₂O was shown not to be an effective emulsion. The following candidate emulsions were shown to be effective at killing/disabling pathogens: 5% Tween 20, 5% Cetylpyridinium Chloride, 10% Glycerol, 60% Soybean Oil, and 20% diH₂O (designated herein as W₂₀5GC5); 1% Cetylpyridinium Chloride, 5% Tween 20, 10% Glycerol, 64% Soybean Oil, and 20% diH₂O (designated herein as W₂₀5GC); 1% Cetylpyridinium Chloride, 5% Tween 20, 8% Ethanol, 64% Olive Oil, and 22% diH₂O (designated herein as W₂₀5EC Olive Oil); 1% Cetylpyridinium Chloride, 5% Tween 20, 8% Ethanol, 64% Flaxseed Oil, and 22% diH₂O (designated herein as W₂₀5EC Flaxseed Oil); 1% Cetylpyridinium Chloride, 5% Tween 20, 8% Ethanol, 64% Corn Oil, and 22% diH₂O (designated herein as W₂₀5EC Corn Oil); 1% Cetylpyridinium Chloride, 5% Tween 20, 8% Ethanol, 64% Coconut Oil, and 22% diH₂O (designated herein as W₂₀5EC Coconut Oil); 1% Cetylpyridinium Chloride, 5% Tween 20, 8% Ethanol, 64% Cottonseed Oil, and 22% diH₂O (designated herein as W₂₀5EC Cottonseed Oil); 8% Dextrose, 5% Tween 10, 1% Cetylpyridinium Chloride, 64% Soybean Oil, and 22% diH₂O (designated herein as W₂₀5C Dextrose); 8% PEG 200, 5% Tween 10, 1% Cetylpyridinium Chloride, 64% Soybean Oil, and 22% diH₂O (designated herein as W₂₀5C PEG 200); 8% Methanol, 5% Tween 10, 1% Cetylpyridinium Chloride, 64% Soybean Oil, and 22% diH₂O (designated herein as W₂₀5C Methanol); 8% PEG 1000, 5% Tween 10, 1% Cetylpyridinium Chloride, 64% Soybean Oil, and 22% diH₂O (designated herein as W₂₀5C PEG 1000); 2% W₂₀5EC, 2% Natrosol 250H NF, and 96% diH₂O (designated herein as 2% W₂₀5EC Natrosol 2, also called 2% W₂₀5EC GEL); 2% W₂₀5EC, 1% Natrosol 250H NF, and 97% diH₂O (designated herein as 2% W₂₀5EC Natrosol 1); 2% W₂₀5EC, 3% Natrosol 250H NF, and 95% diH₂O (designated herein as 2% W₂₀5EC Natrosol 3); 2% W₂₀5EC, 0.5% Natrosol 250H NF, and 97.5% diH₂O (designated herein as 2% W₂₀5EC Natrosol 0.5); 2% W₂₀5EC, 2% Methocel A, and 96% diH₂O (designated herein as 2% W₂₀5EC Methocel A); 2% W₂₀5EC, 2% Methocel K, and 96% diH₂O (designated herein as 2% W₂₀5EC Methocel K); 2% Natrosol, 0.1% X8PC, 0.1× PBS, 5 mM L-alanine, 5 mM Inosine, 10 mM Ammonium Chloride, and diH₂O (designated herein as 0.1% X8PC/GE+2% Natrosol); 2% Natrosol, 0.8% Triton X-100, 0.8% Tributyl Phosphate, 6.4% Soybean Oil, 0.1% Cetylpyridinium Chloride, 0.1× PBS, 5 mM L-alanine, 5 mM Inosine, 10 mM Ammonium Chloride, and diH₂O (designated herein as 10% X8PC/GE+2% Natrosol); 1% Cetylpyridinium Chloride, 5% Tween 20, 8% Ethanol, 64% Lard, and 22% diH₂O (designated herein as W₂₀5EC Lard); 1% Cetylpyridinium Chloride, 5% Tween 20, 8% Ethanol, 64% Mineral Oil, and 22% diH₂O (designated herein as W₂₀5EC Mineral Oil); 0.1% Cetylpyridinium Chloride, 2% Nerolidol, 5% Tween 20, 10% Ethanol, 64% Soybean Oil, and 18.9% diH₂O (designated herein as W₂₀5EC_{0.1}N); 0.1% Cetylpyridinium Chloride, 2% Farnesol, 5% Tween 20, 10% Ethanol, 64% Soybean Oil, and 18.9% diH₂O (designated herein as W₂₀5EC_{0.1}F); 0.1% Cetylpyridinium Chloride, 5% Tween 20, 10% Ethanol, 64% Soybean Oil, and 20.9% diH₂O (designated herein as W₂₀5EC0.1); 10% Cetylpyridinium Chloride, 8% Tributyl Phosphate, 8% Triton X-100, 54% Soybean Oil, and 20% diH₂O (designated herein as X8PC₁₀); 5% Cetylpyridinium Chloride, 8% Triton X-100, 8% Tributyl Phosphate, 59% Soybean Oil, and 20% diH₂O (designated herein as X8PC₅); 0.02% Cetylpyridinium Chloride, 0.1% Tween 20, 10% Ethanol, 70% Soybean Oil, and 19.88% diH₂O (designated herein as W₂₀0.1EC_{0.02}); 1% Cetylpyridinium Chloride, 5% Tween 20, 8% Glycerol, 64% Mobil 1, and 22% diH₂O (designated herein as W₂₀5GC Mobil 1); 7.2% Triton X-100, 7.2% Tributyl Phosphate, 0.9% Cetylpyridinium Chloride, 57.6% Soybean Oil, 0.1× PBS, 5 mM L-alanine, 5 mM Inosine, 10 mM Ammonium Chloride, and 25.87% diH₂O (designated herein as 90% X8PC/GE); 7.2% Triton X-100, 7.2% Tributyl Phosphate, 0.9% Cetylpyridinium Chloride, 57.6% Soybean Oil, 1% EDTA, 5 mM L-alanine, 5 mM Inosine, 10 mM Ammonium Chloride, 0.1× PBS, and diH₂O (designated herein as 90% X8PC/GE EDTA); and 7.2% Triton X-100, 7.2% Tributyl Phosphate, 0.9% Cetylpyridinium Chloride, 57.6% Soybean Oil, 1% Sodium Thiosulfate, 5 mM L-alanine, 5 mM Inosine, 10 mM Ammonium Chloride, 0.1× PBS, and diH₂O (designated herein as 90% X8PC/GE STS).

In preferred embodiments of the present invention, the nanoemulsions are non-toxic (e.g., to humans, plants, or animals), non-irritant (e.g., to humans, plants, or animals), and non-corrosive (e.g., to humans, plants, or animals or the environment), while possessing potency against a broad range of microorganisms including bacteria, fungi, viruses, and spores. While a number of the above described nanoemulsions meet these qualifications, the following description provides a number of preferred non-toxic, non-irritant, non-corrosive, anti-microbial nanoemulsions of the present invention (hereinafter in this section referred to as “non-toxic nanoemulsions”).

In some embodiments the non-toxic nanoemulsions comprise surfactant lipid preparations (SLPs) for use as broad-spectrum antimicrobial agents that are effective against bacteria and their spores, enveloped viruses, and fungi. In preferred embodiments, these SLPs comprises a mixture of oils, detergents, solvents, and cationic halogen-containing compounds in addition to several ions that enhance their biocidal activities. These SLPs are characterized as stable, non-irritant, and non-toxic compounds compared to commercially available bactericidal and sporicidal agents, which are highly irritant and/or toxic.

Ingredients for use in the non-toxic nanoemulsions include, but are not limited to: detergents (e.g., TRITON X-100 (5-15%) or other members of the TRITON family, TWEEN 60 (0.5-2%) or other members of the TWEEN family, or TYLOXAPOL (1-10%)); solvents (e.g., tributyl phosphate (5-15%)); alcohols (e.g., ethanol (5-15%) or glycerol (5-15%)); oils (e.g., soybean oil (40-70%)); cationic halogen-containing compounds (e.g., cetylpyridinium chloride (0.5-2%), cetylpyridinium bromide (0.5-2%)), or cetyldimethylethyl ammonium bromide (0.5-2%)); quaternary ammonium compounds (e.g., benzalkonium chloride (0.5-2%), N-alkyldimethylbenzyl ammonium chloride (0.5-2%)); ions (calcium chloride (1 mM-40 mM), ammonium chloride (1 mM-20 mM), sodium chloride (5 mM-200 mM), sodium phosphate (1 mM-20 mM)); nucleosides (e.g., inosine (50 μM-20 mM)); and amino acids (e.g., L-alanine (50 μM-20 mM)). Emulsions are prepared, for example, by mixing in a high shear mixer for 3-10 minutes. The emulsions may or may not be heated before mixing at 82° C. for 1 hour.

Quaternary ammonium compounds for use in the present include, but are not limited to, N-alkyldimethyl benzyl ammonium saccharinate; 1,3,5-Triazine-1,3,5(2H,4H,6H)-triethanol; 1-Decanaminium, N-decyl-N, N-dimethyl-, chloride (or) Didecyl dimethyl ammonium chloride; 2-(2-(p-(Diisobuyl)cresosxy)ethoxy)ehyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis (2-hydroxyethyl) octyl hydrogen ammonium chloride; dodecyl dimethyl benzyl ammonium chloride; dodecylcarbamoyl methyl dinethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride.

In general, the preferred non-toxic nanoemulsions are characterized by the following: they are approximately 200-800 nm in diameter, although both larger and smaller diameter nanoemulsions are contemplated; the charge depends on the ingredients; they are stable for relatively long periods of time (e.g., up to two years), with preservation of their biocidal activity; they are non-irritant and non-toxic compared to their individual components due, at least in part, to their oil contents that markedly reduce the toxicity of the detergents and the solvents; they are effective at concentrations as low as 0.1%; they have antimicrobial activity against most vegetative bacteria (including Gram-positive and Gram-negative organisms), fungi, and enveloped and nonenveloped viruses in 15 minutes (e.g., 99.99% killing); and they have sporicidal activity in 1-4 hours (e.g., 99.99% killing) when produced with germination enhancers.

The present invention is not limited by the type or strain of orthopox virus used (e.g., in a composition comprising a NE and immunogen (e.g., orthopox virus inactivated by the nanoemulsion). Indeed, each orthopox virus family member alone, or in combination with another family member, may be used to generate a composition comprising a NE and an immunogen (e.g., used to generate an immune response) of the present invention. Orthopox virus family member include, but are not limited to, variola virus, vaccinia virus, cowpox, monkeypox, gergilpox, camelpox, and others. The present invention is not limited by the strain of vaccinia virus used. Indeed, a variety of vaccinia virus strains are contemplated to be useful in the present invention including, but not limited to, classical strains of vaccinia virus (e.g., EM-63, Lister, New York City Board of Health, Elestree, and Temple of Heaven strains), attenuated strains (e.g., Ankara), non-replicating strains, modified strains (e.g., genetically or mechanically modified strains (e.g., to become more or less virulent)), Copenhagen strain, modified vaccinia Ankara, New York vaccinia virus, Vaccinia Virus_{WR} and Vaccinia Virus_{WR-Luc} (See, e.g., Example 1), or other serially diluted strain of vaccinia virus. A composition comprising a NE and immunogen may comprise one or more strains of vaccinia virus and/or other type of orthopox virus. Additionally, a composition comprising a NE and immunogen may comprise one or more strains of vaccinia virus, and, in addition, one or more strains of a non-vaccinia virus immunogen or immunogenic epitope thereof (e.g., a bacteria (e.g., B. anthracis) or immunogenic epitope thereof (e.g., recombinant protective antigen) or a virus (e.g., West Nile virus, Avian Influenza virus, Ebola virus, HSV, HPV, HCV, HIV, etc.) or an immunogenic epitope thereof (e.g., gp120)).

In some embodiments, the immunogen may comprise one or more antigens derived from a pathogen (e.g., orthopox virus). For example, in some embodiments, the immunogen is a purified, recombinant, synthetic, or otherwise isolated protein (e.g., added to the NE to generate an immunogenic composition). Similarly, the immunogenic protein may be a derivative, analogue or otherwise modified (e.g., PEGylated) form of a protein from a pathogen.

The present invention is not limited by the particular formulation of a composition comprising a NE and immunogen of the present invention. Indeed, a composition comprising a NE and immunogen of the present invention may comprise one or more different agents in addition to the NE and immunogen. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a NE and immunogen of the present invention comprises an agent and/or co-factor that enhance the ability of the immunogen to induce an immune response (e.g., an adjuvant). In some preferred embodiments, the presence of one or more co-factors or agents reduces the amount of immunogen required for induction of an immune response (e.g., a protective immune respone (e.g., protective immunization)). In some embodiments, the presence of one or more co-factors or agents can be used to skew the immune response towards a cellular (e.g., T cell mediated) or humoral (e.g., antibody mediated) immune response. The present invention is not limited by the type of co-factor or agent used in a therapeutic agent of the present invention.

Adjuvants are described in general in Vaccine Design--the Subunit and Adjuvant Approach, edited by Powell and Newman, Plenum Press, New York, 1995. The present invention is not limited by the type of adjuvant utilized (e.g., for use in a composition (e.g., pharmaceutical composition) comprising a NE and immunogen). For example, in some embodiments, suitable adjuvants include an aluminium salt such as aluminium hydroxide gel (alum) or aluminium phosphate. In some embodiments, an adjuvant may be a salt of calcium, iron or zinc, or may be an insoluble suspension of acylated tyrosine, or acylated sugars, cationically or anionically derivatised polysaccharides, or polyphosphazenes.

In some embodiments, it is preferred that a composition comprising a NE and immunogen of the present invention comprises one or more adjuvants that induce a Th1-type response. However, in other embodiments, it will be preferred that a composition comprising a NE and immunogen of the present invention comprises one or more adjuvants that induce a Th2-type response.

In general, an immune response is generated to an antigen through the interaction of the antigen with the cells of the immune system. Immune responses may be broadly categorized into two categories: humoral and cell mediated immune responses (e.g., traditionally characterized by antibody and cellular effector mechanisms of protection, respectively). These categories of response have been termed Th1-type responses (cell-mediated response), and Th2-type immune responses (humoral response).

Stimulation of an immune response can result from a direct or indirect response of a cell or component of the immune system to an intervention (e.g., exposure to an immunogen). Immune responses can be measured in many ways including activation, proliferation or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titer; splenomegaly (including increased spleen cellularity); hyperplasia and mixed cellular infiltrates in various organs. Other responses, cells, and components of the immune system that can be assessed with respect to immune stimulation are known in the art.

Although an understanding of the mechanism is not necessary to practice the present invention and the present invention is not limited to any particular mechanism of action, in some embodiments, compositions and methods of the present invention induce expression and secretion of cytokines (e.g., by macrophages, dendritic cells and CD4+ T cells). Modulation of expression of a particular cytokine can occur locally or systemically. It is known that cytokine profiles can determine T cell regulatory and effector functions in immune responses. In some embodiments, Th1-type cytokines can be induced, and thus, the immunostimulatory compositions of the present invention can promote a Th1 type antigen-specific immune response including cytotoxic T-cells. However in other embodiments, Th2-type cytokines can be induced thereby promoting a Th2 type antigen-specific immune response.

Cytokines play a role in directing the T cell response. Helper (CD4+) T cells orchestrate the immune response of mammals through production of soluble factors that act on other immune system cells, including B and other T cells. Most mature CD4+ T helper cells express one of two cytokine profiles: Th1 or Th2. Th1-type CD4+ T cells secrete IL-2, IL-3, IFN-γ, GM-CSF and high levels of TNF-α. Th2 cells express IL-3, IL-4, IL-S, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities whereas those associated with Th2 responses are associated more with allergic responses.

Several factors have been shown to influence skewing of an immune response towards either a Th1 or Th2 type response. The best characterized regulators are cytokines. IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production.

Thus, in some preferred embodiments, the present invention provides a method of stimulating a Th1-type immune response in a subject comprising administering to a subject a composition comprising a NE and an immunogen. However, in other preferred embodiments, the present invention provides a method of stimulating a Th2-type immune response in a subject comprising administering to a subject a composition comprising a NE and an immunogen. In further preferred embodiments, adjuvants can be used (e.g., can be co-administered with a composition of the present invention) to skew an immune response toward either a Th1 or Th2 type immune response. For example, adjuvants that induce Th2 or weak Th1 responses include, but are not limited to, alum, saponins, and SB-As4. Adjuvants that induce Th1 responses include but are not limited to MPL, MDP, ISCOMS, IL-12, IFN-γ, and SB-AS2.

Several other types of Th1-type immunogens can be used (e.g., as an adjuvant) in compositions and methods of the present invention. These include, but are not limited to, the following. In some embodiments, monophosphoryl lipid A (e.g., in particular 3-de-O-acylated monophosphoryl lipid A (3D-MPL)), is used. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. In some embodiments, diphosphoryl lipid A, and 3-0-deacylated variants thereof are used. Each of these immunogens can be purified and prepared by methods described in GB 2122204B, hereby incorporated by reference in its entirety. Other purified and synthetic lipopolysaccharides have been described (See, e.g., U.S. Pat. No. 6,005,099 and EP 0 729 473; Hilgers et al., 1986, Int. Arch. Allergy. Immunol., 79(4):392-6; Hilgers et al., 1987, Immunology, 60(1):141-6; and EP 0 549 074, each of which is hereby incorporated by reference in its entirety). In some embodiments, 3D-MPL is used in the form of a particulate formulation (e.g., having a small particle size less than 0.2 μm in diameter, described in EP 0 689 454, hereby incorporated by reference in its entirety).

In some embodiments, saponins are used as an immunogen (e.g.,Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit Rev Ther Drug Carrier Syst, 1996, 12 (1-2):1-55; and EP 0 362 279, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146,431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety.

In some embodiments, an immunogenic oligonucleotide containing umnethylated CpG dinucleotides (“CpG”) is used as an adjuvant in the present invention. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Imnunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

Although an understanding of the mechanism is not necessary to practice the present invention and the present invention is not limited to any particular mechanism of action, in some embodiments, the presence of one or more CpG oligonucleotides activate various immune subsets including natural killer cells (which produce IFN-γ) and macrophages. In some embodiments, CpG oligonucleotides are formulated into a composition of the present invention for inducing an immune response. In some embodiments, a free solution of CpG is co-administered together with an antigen (e.g., present within a NE solution (See, e.g., WO 96/02555; hereby incorporated by reference). In some embodiments, a CpG oligonucleotide is covalently conjugated to an antigen (See, e.g., WO 98/16247, hereby incorporated by reference), or formulated with a carrier such as aluminium hydroxide (See, e.g., Brazolot-Millan et al., Proc. Natl. Acad Sci., USA, 1998, 95(26), 15553-8).

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. Coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (See, e.g., WO93/13202 and WO92/19265, each of which is hereby incorporated by reference), and other immunogenic substances (e.g., that enhance the effectiveness of a composition of the present invention) are used with a composition comprising a NE and immunogen of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants may be added to a composition comprising a NE and an immunogen, or, the adjuvant may be formulated with carriers, for example liposomes, or metallic salts (e.g., aluminium salts (e.g., aluminium hydroxide)) prior to combining with or co-administration with a composition comprising a NE and an immunogen.

In some embodiments, a composition comprising a NE and an immunogen comprises a single adjuvant. In other embodiments, a composition comprising a NE and an immunogen comprises two or more adjuvants (See, e.g., WO 94/00153; WO 95/17210; WO 96/33739; WO 98/56414; WO 99/12565; WO 99/11241; and WO 94/00153, each of which is hereby incorporated by reference in its entirety).

In some embodiments, a composition comprising a NE and an immunogen of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. Although an understanding of the mechanism is not necessary to practice the present invention and the present invention is not limited to any particular mechanism of action, in some embodiments, use of a mucoadhesive (e.g., in a composition comprising a NE and immunogen) enhances induction of an immune response in a subject (e.g., administered a composition of the present invention) due to an increase in duration and/or amount of exposure to an immunogen that a subject experiences when a mucoadhesive is used compared to the duration and/or amount of exposure to an immunogen in the absence of using the mucoadhesive.

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising a NE and an immunogen of the present invention can be used therapeutically (e.g., to enhance an immune response) or as a prophylactic (e.g., for immunization (e.g., to prevent signs or symptoms of disease)). A composition comprising a NE and an immunogen of the present invention can be administered to a subject via a number of different delivery routes and methods.

For example, the compositions of the present invention can be administered to a subject (e.g., mucosally (e.g., nasal mucosa, vaginal mucosa, etc.)) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal surface); being placed on or impregnated onto a nasal and/or vaginal applicator and applied; being applied by a controlled-release mechanism; being applied as a liposome; or being applied on a polymer.

In some preferred embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal and rectal techniques), as well as European Publication No. 517,565 and Illum et al., J. Controlled Rel., 1994, 29:133-141 (e.g.,for techniques of intranasal administration), each of which is hereby incorporated by reference in its entirety). Alternatively, the compositions of the present invention may be administered dermally or transdermally, using standard techniques (See, e.g., Remington: The Science arid Practice of Pharmacy, Mack Publishing Company, Easton, Pa., 19th edition, 1995). The present invention is not limited by the route of administration.

Although an understanding of the mechanism is not necessary to practice the present invention and the present invention is not limited to any particular mechanism of action, in some embodiments, mucosal vaccination is the preferred route of administration as it has been shown that mucosal administration of antigens has a greater efficacy of inducing protective immune responses at mucosal surfaces (e.g., mucosal immunity), the route of entry of many pathogens. In addition, mucosal vaccination, such as intranasal vaccination, may induce mucosal immunity not only in the nasal mucosa, but also in distant mucosal sites such as the genital mucosa (See, e.g., Mestecky, Journal of Clinical Immunology, 7:265-276, 1987). More advantageously, in further preferred embodiments, in addition to inducing mucosal immune responses, mucosal vaccination also induces systemic immunity. In some embodiments, non-parenteral administration (e.g., muscosal administration of vaccines) provides an efficient and convenient way to boost systemic immunity (e.g., induced by parenteral or mucosal vaccination (e.g., in cases where multiple boosts are used to sustain a vigorous systemic immunity)).

In some embodiments, a composition comprising a NE and an immunogen of the present invention may be used to protect or treat a subject susceptible to, or suffering from, disease by means of administering a composition of the present invention via a mucosal route (e.g., an oral/alimentary or nasal route). Alternative mucosal routes include intravaginal and intra-rectal routes. In preferred embodiments of the present invention, a nasal route of administration is used, termed “intranasal administration” or “intranasal vaccination” herein. Methods of intranasal vaccination are well known in the art, including the administration of a droplet or spray form of the vaccine into the nasopharynx of a sujbect to be immunized. In some embodiments, a nebulized or aerosolized composition comprising a NE and immunogen is provided. Enteric formulations such as gastro resistant capsules for oral administration, suppositories for rectal or vaginal administration also form part of this invention. Compositions of the present invention may also be administered via the oral route. Under these circumstances, a composition comprising a NE and an immunogen may comprise a pharmaceutically acceptable excipient and/or include alkaline buffers, or enteric capsules. Formulations for nasal delivery may include those with dextran or cyclodextran and saponin as an adjuvant.

Compositions of the present invention may also be administered via a vaginal route. In such cases, a composition comprising a NE and an immunogen may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, a composition comprising a NE and an immunogen may comprise excipients and/or waxes and polymers known in the art for forming rectal suppositories.

In some embodiments, the same route of administration (e.g., mucosal administration) is chosen for both a priming and boosting vaccination. In some embodiments, multiple routes of administration are utilized (e.g., at the same time, or, alternatively, sequentially) in order to stimulate an immune response (e.g., using a composition comprising a NE and immunogen of the present invention).

For example, in some embodiments, a composition comprising a NE and an immunogen is administered to a mucosal surface of a subject in either a priming or boosting vaccination regime. Alternatively, in some embodiments, a composition comprising a NE and an immunogen is administered systemically in either a priming or boosting vaccination regime. In some embodiments, a composition comprising a NE and an immunogen is administered to a subject in a priming vaccination regimen via mucosal administration and a boosting regimen via systemic administration. In some embodiments, a composition comprising a NE and an immunogen is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal or intravenous administration. A composition comprising a NE and an immunogen may be used for both prophylactic and therapeutic purposes.

In some embodiments, compositions of the present invention are administered by pulmonary delivery. For example, a composition of the present invention can be delivered to the lungs of a subject (e.g., a human) via inhalation (e.g., thereby traversing across the lung epithelial lining to the blood stream (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. 111, pp. 206-212; Smith, et al. J. Clin. Invest. 1989;84:1145-1146; Oswein, et al. “Aerosolization of Proteins”, 1990; Proceedings of Symposium on Respiratory Drug Delivery II Keystone, Colorado; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for dispensing of the therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising a NE and an immunogen of the present invention may be used to protect and/or treat a subject susceptible to, or suffering from, a disease by means of administering a compositions comprising a NE and an immunogen by mucosal, intramuscular, intraperitoneal, intradermal, transdermal, pulmonary, intravenous, subcutaneous or other route of administration described herein. Methods of systemic administration of the vaccine preparations may include conventional syringes and needles, or devices designed for ballistic delivery of solid vaccines (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). The present invention may also be used to enhance the immunogenicity of antigens applied to the skin (transdermal or transcutaneous delivery, See, e.g., WO 98/20734 ; WO 98/28037, each of which are hereby incorporated by reference). Thus, in some embodiments, the present invention provides a delivery device for systemic administration, pre-filled with the vaccine composition of the present invention.

The present invention is not limited by the type of subject administered (e.g., in order to stimulate an immune response (e.g., in order to generate protective immunity (e.g., mucosal and/or systemic immunity))) a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism. In some embodiments, the human subjects are subjects that are more likely to receive a direct exposure to pathogenic microorganisms or that are more likely to display signs and symptoms of disease after exposure to a pathogen (e.g., subjects in the armed forces, government employees, frequent travelers, persons attending or working in a school or daycare, health care workers, an elderly person, an immunocompromised person, and emergency service employees (e.g., police, fire, EMT employees)). In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease) and/or to prevent or reduce the risk of disease spread from animals (e.g., birds, cattle, sheep, pigs, etc.) to humans. In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition of the present invention may be formulated for administration by any route, such as mucosal, oral, topical, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxyethyl) pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments, pharmaceutical compositions of the present invention may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the NE and immunogen of the formulation. In some embodiments, immunostimulatory compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a NE and an immunogen is co-administered with one or more antibiotics. For example, one or more antibiotics may be administered with, before and/or after administration of a composition comprising a NE and an immunogen. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines

There are an enormous amount of antimicrobial agents currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a NE and an immunogen with one or more additional active and/or immunostimulatory agents (e.g., a composition comprising a NE and a different immnogen, an antibiotic, anti-oxidant, etc.). Indeed, it is a further aspect of this invention to provide methods for enhancing prior art immunostimulatory methods (e.g., immunization methods) and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, adjuvants, etc.) can be any of the well-known agents in the art, including, but not limited to, those that are currently in clinical use.

In some embodiments, a composition comprising a NE and immunogen is administered to a subject via more than one route. For example, a subject that would benefit from having a protective immune response (e.g., immunity) towards a pathogenic microorganism may benefit from receiving mucosal administration (e.g., nasal administration or other mucosal routes described herein) and, additionally, receiving one or more other routes of administration (e.g., parenteral or pulmonary administration (e.g., via a nebulizer, inhaler, or other methods described herein). In some preferred embodiments, administration via mucosal route is sufficient to induce both mucosal as well as systemic immunity towards an immunogen or organism from which the immunogen is derived. In other embodiments, administration via multiple routes serves to provide both mucosal and systemic immunity. Thus, although an understanding of the mechanism is not necessary to practice the present invention and the present invention is not limited to any particular mechanism of action, in some embodiments, it is contemplated that a subject administered a composition of the present invention via multiple routes of administration (e.g., immunization (e.g., mucosal as well as airway or parenteral administration of a composition comprising a NE and immunogen of the present invention) may have a stronger immune response to an immunogen than a subject administered a composition via just one route.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In preferred embodiments, a composition comprising a NE and an immunogen of the present invention comprises a suitable amount of the immunogen to induce an immune response in a subject when administered to the subject. In preferred embodiments, the immune response is sufficient to provide the subject protection (e.g., immune protection) against a subsequent exposure to the immunogen or the microorganism (e.g., bacteria or virus) from which the immunogen was derived. The present invention is not limited by the amount of immunogen used. In some preferred embodiments, the amount of immunogen (e.g., virus or bacteria neutralized by the NE, or, recombinant protein) in a composition comprising a NE and immunogen (e.g., for use as an immunization dose) is selected as that amount which induces an immunoprotective response without significant, adverse side effects. The amount will vary depending upon which specific immunogen or combination thereof is/are employed, and can vary from subject to subject, depending on a number of factors including, but not limited to, the species, age and general condition (e.g., health) of the subject, and the mode of administration. Procedures for determining the appropriate amount of immunogen administered to a subject to elicit an immune response (e.g., a protective immune response (e.g., protective immunity)) in a subject are well known to those skilled in the art.

In some embodiments, it is expected that each dose (e.g., of a composition comprising a NE and an immunogen (e.g., administered to a subject to induce an immune response (e.g., a protective immune response (e.g., protective immunity))) comprises 0.05-5000 μg of each immunogen (e.g., recombinant and/or purified protein), in some embodiments, each dose will comprise 1-500 μg, in some embodiments, each dose will comprise 350-750 μg, in some embodiments, each dose will comprise 50-200 μg, in some embodiments, each dose will comprise 25-75 μg of immunogen (e.g., recombinant and/or purified protein). In some embodiments, each dose comprises an amount of the immunogen sufficient to generated an immune response. An effective amount of the immunogen in a dose need not be quantified, as long as the amount of immunogen generates an immune response in a subject when administered to the subject. An optimal amount for a particular administration (e.g., to induce an immune (e.g., a protective immune response (e.g., protective immunity))) can be ascertained by one of skill in the art using standard studies involving observation of antibody titers and other responses in subjects.

In some embodiments, it is expected that each dose (e.g., of a composition comprising a NE and an immunogen (e.g., administered to a subject to induce and immune response)) is from 0.001 to 15% or more (e.g., 0.001-10%, 0.5-5%, 1-3%, 2%, 6%,10%,15% or more) by weight immunogen (e.g., neutralized bacteria or virus, or recombinant and/or purified protein). In some embodiments, an initial or prime administration dose contains more immunogen than a subsequent boost dose In some embodiments, when a NE of the present invention is utilized to inactivate a live microorganism (e.g., virus (e.g., a purified strain of an orthopox virus (e.g., vaccinia virus))), it is expected that each dose (e.g., administered to a subject to induce and immune response)) comprises between 10 and 10⁹ pfu of the virus per dose; in some embodiments, each dose comprises between 10⁵ and 10⁸ pfu of the virus per dose; in some embodiments, each dose comprises between 10³ and 10⁵ pfu of the virus per dose; in some embodiments, each dose comprises between 10² and 10⁴ pfu of the virus per dose; in some embodiments, each dose comprises 10 pfu of the virus per dose; in some embodiments, each dose comprises 10² pfu of the virus per dose; and in some embodiments, each dose comprises 10⁴ pfu of the virus per dose. In some embodiments, each dose comprises more than 10⁹ pfu of the virus per dose. In some preferred embodiments, each dose comprises 10³ pfu of the virus per dose.

The present invention is not limited by the amount of NE used to inactivate a live microorganism (e.g., orthopox virus (e.g., vaccinia virus)). In some embodiments, a 0.1%-5% NE solution is used, in some embodiments, a 5%-20% NE solution is used, in some embodiments, a 20% NE solution is used, and in some embodiments, a NE solution greater than 20% is used order to inactivate a pathogenic microorganism. In preferred embodiments, a 10% NE solution is used.

Similarly, the present invention is not limited by the duration of time a live microorganism is incubated in a NE of the present invention in order to become inactivated. In some embodiments, the microorganism is incubated for 1-3 hours in NE. In some embodiments, the microorganism is incubated for 3-6 hours in NE. In some embodiments, the microorganism is incubated for more than 6 hours in NE. In preferred embodiments, the microorganism is incubated for 3 hours in NE (e.g., a 10% NE solution). In some embodiments, the incubation is carried out at 37° C. In some embodiments, the incubation is carried out at a temperature greater than or less than 37° C. The present invention is also not limited by the amount of microorganism used for inactivation. The amount of microorganism may depend upon a number of factors including, but not limited to, the total amount of immunogenic composition (e.g., NE and immunogen) desired, the concentration of solution desired (e.g., prior to dilution for administration), the microorganism and the NE. In some preferred embodiments, the amount of microorganism used in an inactivation procedure is that amount that produces the desired amount of immunogen (e.g., as described herein) to be administered in a single dose (e.g., diluted from a concentrated stock) to a subject (See, e.g., Example 1).

In some embodiments, a composition comprising a NE and an immunogen of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of the NE and immunogen present in the concentrated composition. In some preferred embodiments, a subject is administered in a single dose a composition comprising 1% of the NE and immunogen present in the concentrated composition. Concentrated compositions are contemplated to be useful in a setting in which large numbers of subjects may be administered a composition of the present invention (e.g., an immunization clinic, hospital, school, etc.). In some embodiments, a composition comprising a NE and an immunogen of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

In some embodiments, when a NE of the present invention is utilized to neutralize a live microorganism (e.g., virus (e.g., a purified strain of a pathogenic virus)), it is expected that each dose (e.g., of a composition comprising a NE and an immunogen, wherein the immunogen is neutralized by the NE (e.g., administered to a subject to induce and immune response)) comprises a tissue culture infectious dose₅₀ (TCID₅₀; the quantity of virus in a specified suspension volume (e.g., 0.1 ml) that will infect 50% of a number (n) of cell culture microplate wells, or tubes, See e.g., J Infect Dis. (2004)190:1962-1969) of 0.1-500 of the virus; in some embodiments, each dose comprises a TCID₅₀ of 10-250 of the virus; and in some embodiments, each dose comprises a TCID₅₀ of 100-200 of the virus.

In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second and/or third administration. Although an understanding of the mechanism is not necessary to practice the present invention and the present invention is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

A composition comprising an immunogen of the present invention finds use where the nature of the infectious and/or disease causing agent (e.g., for which protective immunity is sought to be elicited) is known, as well as where the nature of the infectious and/or disease causing agent is unknown (e.g., in emerging disease (e.g., of pandemic proportion (e.g., influenza or other outbreaks of disease))). For example, the present invention contemplates use of the compositions of the present invention in treatment of or prevention of (e.g., via immunization with an infectious and/or disease causing agent neutralized via a NE of the present invention) infections associated with an emergent infectious and/or disease causing agent yet to be identified (e.g., isolated and/or cultured from a diseased person but without genetic, biochemical or other characterization of the infectious and/or disease causing agent).

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions, immunogens, and other components and for screening for new components. Thus, it is not intended that the present invention be limited to any particular subject and/or application setting.

The formulations can be tested in vivo in a number of animal models developed for the study of mucosal and other routes of delivery. As is readily apparent, the compositions of the present invention are useful for preventing and/or treating a wide variety of diseases and infections caused by viruses, bacteria, parasites, and fungi, as well as for eliciting an immune response against a variety of antigens. Not only can the compositions be used prophylactically or therapeutically, as described above, the compositions can also be used in order to prepare antibodies, both polyclonal and monoclonal (e.g., for diagnostic purposes), as well as for immunopurification of an antigen of interest. If polyclonal antibodies are desired, a selected mammal, (e.g., mouse, rabbit, goat, horse, etc.) can be immunized with the compositions of the present invention. The animal is usually boosted 2-6 weeks later with one or more—administrations of the antigen. Polyclonal antisera can then be obtained from the immunized animal and used according to known procedures (See, e.g., Jurgens et al., J. Chrom. 1985, 348:363-370).

In some embodiments, bioluminescence imaging (BLI) can be used to characterize protection of a subject from smallpox (e.g., after administration of a composition of the present invention). BLI provides multiple advantages for investigating viral infections and host immunity in mouse models (See, e.g., Cook and Griffin, (2003), J Virol 77(9), 5333-5338; Luker et al., Virology. 2005, 341(2):284-300; Luker et al., 2002 J Virol 76(23), 12149-12161). Sites of infection and relative amounts of virus in various anatomic sites can be detected and quantified, allowing the spatial and temporal progression of infection to be monitored in a subject (e.g., a mouse). BLI provides enhanced information about the extent of viral replication in living subjects relative to other in vivo assays of disease progression, such as weight loss or external signs of disease. In particular, BLI can be used to establish biologically relevant relationships among vaccination strategies, production of neutralizing antibodies, and viral dissemination, and reinforce the unique efficacy of these formulations.

In some embodiments, the present invention provides a kit comprising a composition comprising a NE and an immunogen. In some embodiments, the kit further provides a device for administering the composition. The present invention is not limited by the type of device included in the kit. In some embodiments, the device is configured for nasal application of the composition of the present invention (e.g., a nasal applicator (e.g., a syringe) or nasal inhaler or nasal mister). In some embodiments, a kit comprises a composition comprising a NE and an immunogen in a concentrated form (e.g., that can be diluted prior to administration to a subject).

In some embodiments, all kit components are present within a single container (e.g., vial or tube). In some embodiments, each kit component is located in a single container (e.g., vial or tube). In some embodiments, one or more kit component are located in a single container (e.g., vial or tube) with other components of the same kit being located in a separate container (e.g., vial or tube). In some embodiments, a kit comprises a buffer. In some embodiments, the kit further comprises instructions for use.

EXPERIMENTAL

The following examples are provided in order to demonstrate and further illustrate certain preferred embodiments and aspects of the present invention and are not to be construed as limiting the scope thereof.

### EXAMPLE 1

### Materials and Methods

Animals. Pathogen-free, 5 to 6-week-old, female Balb/c mice were purchased from Charles River Laboratories. Vaccination groups were housed separately, five animals to a cage, in accordance with the American Association for Accreditation of Laboratory Animal Care standards. All procedures involving mice were performed according to the University Committee on Use and Care of Animals (UCUCA) at the University of Michigan.

Viruses. Two exemplary vaccinia viruses (VV) were used during the development of the present invention, VV_{WR} and VV_{WR-Luc}. VV_{WR} (NIH TC-adapted) was obtained from the American Type Culture Collection (ATCC). Recombinant VV_{WR-Luc} expresses firefly luciferase from the p7.5 early/late promoter and has been described (See, e.g., Luker et al., Virology. 2005, 341(2):284-300). VV_{WR-Luc} is not attenuated in vitro or in vivo because the virus was constructed with a method that does not require deletion of any viral genes (See, e.g., Blasco and Moss (1995). Gene 158(2), 157-162; Luker et al., Virology. 2005, 341(2):284-300).

Stocks of all viruses were generated using the method of Lorenzo et al (See, e.g., Lorenzo et al., Methods Mol Biol. 2004; 269:15-30) with some modification. Virus was propogated on Vero cells infected at a multiplicity of infection of 0.5. Cells were harvested at 48 to 72 h and virus was isolated from culture supernatants and cells lysates. Cell lysates were obtained by rapidly freeze-thawing the cell pellet followed by homogenization in Dounce homogenizer in 1 mM Tris pH 9.0. Cell debris was removed by centrifugation at 2000 rpm. The purified virus stocks were obtained from clarified supernatants by layering on 4% to 40% sucrose gradients which were centrifuged for 1 hr at 25,000×g. Turbid bands, containing viral particles, were collected, diluted in 1 mM Tris pH 9 and then concentrated by 1 hr centrifugation at 25,000×g. Viral pellets were re-suspended in 1 mM Tris pH 9 and stored frozen at −80° C. as virus stock. The VV_{WR} stocks were titered on Vero cells (See, e.g., Myc et al., Vaccine. 21:3801-3814). VV_{WR} has identical surface proteins as the native strain, but expresses luciferase protein during infection. This allowed for a sensitive cytotoxicity and morbidity assessment and the monitoring of the viral infection in animals with imaging techniques. Comparison of the serological response in VV_{WR} immunized animals either by ELISA, Western blot or virus neutralization assays showed no difference in titer to either VV_{WR} or VV_{WR-Luc}.

Nanoemulsion (NE). NE (W₂₀5EC) was obtained from NANOBIO Corporation, Ann Arbor, Mich. (See U.S. Pat. No. 6,015,832 issued to NANOBIO Corporation (Ann Arbor, Mich.), herein incorporated by reference in its entirety). The nanoemulsion was manufactured by emulsification of cetyl pyridum chloride (1%), Tween 20 (5%) and Ethanol (8%) in water with soybean oil (64%) using a high speed emulsifier. Resultant droplets have a mean particle size of 300±25 nm in diameter. W₂₀5EC has been formulated with surfactants and food substances considered “Generally Recognized as Safe” (GRAS) by the FDA. W₂₀5EC can be economically manufactured under Good Manufacturing Practices (GMP) and is stable for at least 18 months at 40° C.

Preparation of the NE-based vaccine. Vaccinia virus (VV) neutralization data generated during the development of the present invention indicated that 1 hr incubation with 10% NE or 0.1% formalin was sufficient for inactivation of the virus (e.g., six log VV titer reduction). On the basis of these results several formulations (e.g., compositions) for inducing an immune response (e.g., vaccine formulations) were produced for animal immunization. The compositions (e.g., for stimulating an immune response) were prepared as follow: To assure complete virus neutralization (e.g., virus inactivation) for the NE-killed VV, samples containing 1×10³ pfu to 5×10⁵ pfu per dose of VV were incubated for 3 hrs at 37° C. in 10% W₂₀5EC NE, and were subsequently diluted to 1% NE for intranasal instillation (e.g., 1×10³ to 1×10⁵ pfu per dose). For the vaccine formulations containing formalin-killed virus, the formalin (SIGMA) inactivation of VV was performed at RT for 3 hrs in 0.1% formalin. Formalin-killed virus was diluted in either saline or 1% NE to 10³ or 10⁵ pfu per dose to reduce the formalin to nontoxic concentrations for intranasal immunization. For every formulation in each experiment, virus inactivation by either NE or formalin was confirmed in vitro by infecting Vero cells, followed with two subsequent passages of culture supernatants after 3-4 days of incubation. None of the control infections showed a presence of viral plaques. Additionally, PCR-detection assays of viral DNA in Vero cells and lungs of treated animals were performed as described below to confirm the absence of live, replicating virus.

Immunization. Samples of pre-immune serum were collected from mice prior to initial immunization. All animals were anesthetized with isoflurane and vaccinated (e.g., with 10-15 μl of vaccine formulation per nare) using a pipette tip. Emulsion was administered slowly to minimize the swallowing of material. After vaccination, animals were observed for adverse reactions. Specific anti-VV antibody response was measured in blood samples 3 weeks after the initial (e.g., prime) administration (e.g., immunization) and at two to three week intervals after the second and third administrations (e.g., immunizations) when additional administrations were performed.

Immunization by scarification was performed in anesthetized mice by superficial scarification at the base of the tail. Before the procedure, hair was removed by a clipper to expose approximately 0.5-0.7 square centimeters and the naked skin was disinfected with 70% ethanol. A sterile bifurcate needle was used to superficially abrade the epidermis and 1×10⁵ pfu dose of live VV_{WR} was applied in 10 μl PBS. Animals were immobilezed for up to 10 minutes to ensure virus absorption into the skin.

Bioluminescence imaging. Bioluminescence imaging was performed with a cryogenically-cooled CCD camera (IVIS) as described elsewhere (See, e.g., Luker et al., (2002). J Virol 76(23), 12149-12161; Cook and Griffin, (2003). J Virol 77(9), 5333-5338). Data for photon flux were quantified by region-of-interest (ROI) analysis of the head, chest and abdomen of infected mice. Background photon flux from an uninfected mouse injected with luciferin was subtracted from all measurements.

Collection of blood, bronchial alveolar lavage (BAL) and splenocytes. Blood samples were obtained from the saphenous vein at various time points during the course of trials conducted during the development of the present invention. Final samples were obtained by cardiac puncture from euthanized, premorbid mice. Serum was obtained from blood by centrifugation at 1500×g for 5 minutes after the blood coagulated for 30-60 minutes at room temperature. Serum samples were stored at −20° C. until used.

BAL fluid was obtained from mice euthanized by isoflurane inhalation. After the trachea was dissected, a 22 gauge catheter (Angiocath, B-D) attached to a 1 ml syringe was inserted into the trachea. The lungs were infused twice with 0.5 ml of PBS containing 10 μM DTT and 0.5 mg/ml aprotinin. Approximately 1.0 ml of aspirate was recovered with a syringe. BAL samples were stored at −20° C. until analyzed.

Murine splenocytes were mechanically isolated to obtain single-cell suspension in PBS. Red blood cells (RBC) were removed by lysis with ACK buffer (150 mM NH₄Cl, 10 mM KHCO₃, 0.1 mM Na₂EDTA), and the remaining cells washed twice in PBS. For antigen-specific proliferation or cytokine expression assays, splenocytes (2-4×10⁶/ml) were resuspended in RPMI 1640 medium supplemented with 5% FBS, 200 nM L-glutamine, and penicillin/streptomycin (100 U/ml and 100 μg/ml).

PCR detection of viral DNA. Forward primer (SEQ ID NO. 1: 5′-ATG ACA CGA TTG CCA ATA C 3′) and reverse primer (SEQ ID NO. 2: 5′-CTA GAC TTT GTT TTC TG 3′) were used (See, e.g., Ropp et al., J. Clin. Microbiol., 1995: 2069-2076). These primers are for conserved regions of the HA gene of all orthopox viruses (e.g., VV) and were synthesized by Integrated DNA Technologies (IDT, Coralville, Iowa). DNA was isolated from Vero cells or from lung tissue homogenates with Trireagent per the manufacturer's protocol (MRC, Cincinnati, Ohio). PCR amplification was performed with 10 μg of total cell or lung DNA using 0.5 μM of each primer, 0.2 mM of each dNTP, 2.5 mM of MgCl₂, and 0.1 U/μl of Taq DNA Polymerase (ROCHE Molecular Biochemicals, Indianapolis, Ind.). PCR reactions were carried out in a total volume of 20 μl, incubated at 94° C. for 1 min, followed by 25 cycles with annealing at 55° C., extension at 72° C. and denaturation at 94° C. PCR product analysis was performed using electrophoresis on 1% agarose gel in Tris-borate buffer for electrophoresis and ethidium bromide for DNA staining. Analysis was performed using a photoimaging camera and software from BioRad (Hercules, Calif.). Purified VV DNA (1 ng) mixed with lung DNA served as a positive control. Gel analysis was performed using a photoimaging camera and software from BIORAD (Hercules, Calif.).

Specific anti-virus IgA and IgG determination. Mouse anti-vaccinia antibodies were determined by ELISA. Microtiter 96-well flat bottom NUNC-PolySorp polystyrene plates were coated with a dilution of infected Vero cells lysate containing at least 5×10⁴ pfu/well of vaccinia virus in PBS. Plates were incubated overnight at 4° C. and fixed with 50% mixture of ethanol/acetone (EtOH/acetone) for 1 hour at −20° C. After the fixing solution was removed, plates were washed twice with PBS containing 0.001% Tween 20 and then blocked for 1 hour at 37° C. with 1% non-fat dry milk in PBS containing 0.2% Tween 20. Mouse sera or BAL fluid were serially diluted in PBS with 0.1% BSA, 100 μl aliqots were added to wells, and the plates were incubated for 2 hours at 37° C. Plates were washed three times with PBS-0.05% Tween 20, followed by 1 hour incubation with either anti-mouse IgG or anti-mouse IgA alkaline phosphatase (AP)-conjugated antibodies, and then washed three times. The colorimetric reaction was performed with AP substrate SIGMAFAST (SIGMA, St. Louis, Mo.) according to the manufacturer's protocol. Spectrophdtometric readouts were done using the SPECTRAMAX 340 ELISA reader (MOLECULAR DEVICES, Sunnyvale, Calif.) at 405 nm and reference wavelength of 690 nm. The endpoint titers and antibody concentrations were calculated as the serum dilution resulting in an absorbance greater than two standard deviations above the absorbance in control wells. The IgG antibody concentration was calculated according to the logarithmic transformation of the linear portion of the standard curve generated with the AP-conjugated anti-IgG antibody and multiplied by the serum dilution factor. The serum antibody concentrations are presented as a mean value ± standard error (sem). Serum from the naive mice was used as a control for non-specific absorbance.

Anti-VV IgG antibody activity targeted toward alcohol denaturized versus formalin-alkylated viral epitopes was measured using ELISA, as described above with a few modifications. The 96 well plates were coated with 1×10⁵ pfu/well of purified vaccinia virus and incubated overnight at 4° C. After virus was removed, wells were treated for 1 hour with either 50% EtOH/acetone at −20° C. or with 1% formalin solution in PBS at 4° C. Plates were washed and blocked as described above. Pooled sera from mice immunized with various formulations of vaccine (VV/NE, VV/Fk/NE, VV/Fk) and sera from mice which survived sub-lethal infection with live vaccinia virus (VV live) were serially diluted in 0.1% BSA, and 100 μL aliquots were added to EtOH/acetone and to the formalin-fixed wells. The assay was performed as describe above for the anti-vaccinia IgG determination. The optical density (OD) values at 405 nm were compared between EtOH/acetone and formalin-fixed viral antigens. The differences in the activity of anti-vaccinia antibodies were evaluated by the ratio of IgG titers on EtOH/acetone versus formalin at the same OD 405 nm value.

Neutralizing antibodies. Neutralizing antibodies were determined with both a standard plaque reduction assay (PRA) (See, e.g., Newman et al., J. Chem. Microbiol. 2003, 3154-3157) and the inhibition of luciferase activity using recombinant VV_{WR-LUC}. The PRA was conducted by mixing 10 μl of heat-inactivated mouse serum in serial, two-fold dilutions with 10 μl of serum-free RPMI medium containing 200-300 pfu of VV. Sera were incubated 6 hr at 37° C. and subsequently placed in 0.5 ml of serum-free medium an overlaid on Vero cell monolayer. After 1 hr incubation, virus/serum inocula were removed and a fresh medium was placed on the cell monolayers. After 48 to 72 hrs, cells were fixed and stained with 0.1% crystal violet. Plaques were counted by two independent observers and the neutralization titer calculated using non-immune serum as a control.

For the assessment of neutralization titer with VV_{WR-Luc}, 10 μl of heat-inactivated mouse serum in serial, two-fold dilutions were mixed with 10 μl of serum-free RPMI medium containing 2×10³ pfu of virus. As in the PRA based neutralization assay, samples were incubated for 6 hr at 37° C., resuspended in 100 μl of serum-free RPMI and incubated for 1 hr with Vero cells in 24 well plates. After 24-36 hrs, infected cells were lysed and virus-dependent luciferase activity was assessed by the luciferase assay described above. Neutralization titers (NT₅₀) were calculated from the luciferase inhibition curves using non-immune sera and virus in PBS as controls. Correlations between PRA and luciferase inhibition activity was made for each sample.

Vaccinia specific cytokine expression in splenocytes. Spleens from vaccinated mice were harvested 12 weeks after initial vaccination. Splenocytes were obtained from mechanically disrupted spleens and suspended at 3×10⁶ cells/ml in RPMI 1640 supplemented with 5% FBS, L-glutamine and penicillin/streptomycin. Cells were incubated with either 1×10³ or 1×10⁴ pfu per well of vaccinia virus for 72 hours at 37° C. Cell culture supernatants were harvested and analyzed for cytokine production. PHA-P (1 μg per well) was incubated with the cells as a positive control. The IFN-γ concentrations in splenocyte supernatants were determined using QUANTIKINE M ELISA kits (R&D SYSTEMS Inc., Minneapolis, Minn.) according to the manufacturer's directions.

Vaccinia virus challenge. Immunized mice were challenged with live vaccinia virus to evaluate the effectiveness of the vaccine. Serum samples were collected two days before the vaccinia challenge and animals were weighed on the day of the challenge. Aliquots of purified recombinant VV_{WR} or VV_{WR-Luc} (sonicated and titered before use) were thawed and diluted in saline the day of the challenge. Mice were anesthetized by inhalation of isoflurane and challenged intranasally with a 20 μl suspension of 2×10⁶ pfu live VV_{WR-Luc} corresponding to 10×LD₅₀, or with live VV_{WR} doses ranging from 1×10⁷ to 3.2×10³ in 5 fold dilutions. Weight and body temperature were measured daily for 3 weeks following challenge. Mice that demonstrated a 30% loss in initial body weight were euthanized. Lethal dose (LD₅₀) and the infectious dose (ID50) calculations were based on the animals death rates, and on the core body temperature and body weight loss, respectively (See, e.g., Reed and Muench, Am J Hyg 1938;27:493-7). Index of protection against lethal challenge (IP_{LD}) was calculated as follows: EPLD=log₁₀ Maximum dose−log₁₀LD₅₀ controls. Similarly, index of protection against infection (IP_{ID}) was calculated as: IP_{ID}=log₁₀ID50 vaccinated−log10LD₅₀ controls.

Statistical analysis. Statistical analysis of the results was preformed using ANOVA, and Student's T-test for the determination of the p value.

### EXAMPLE 2

### Nasal Immunization with Nanoemulsion-Inactivated Vaccinia Virus Results in the Induction Specific Systemic IgG Response

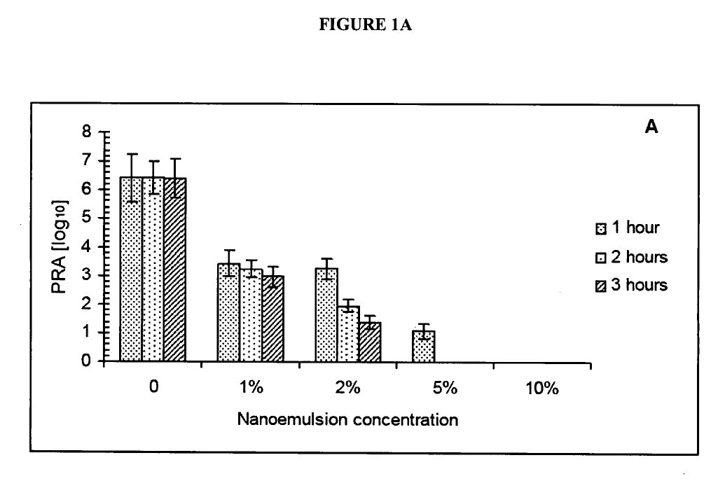

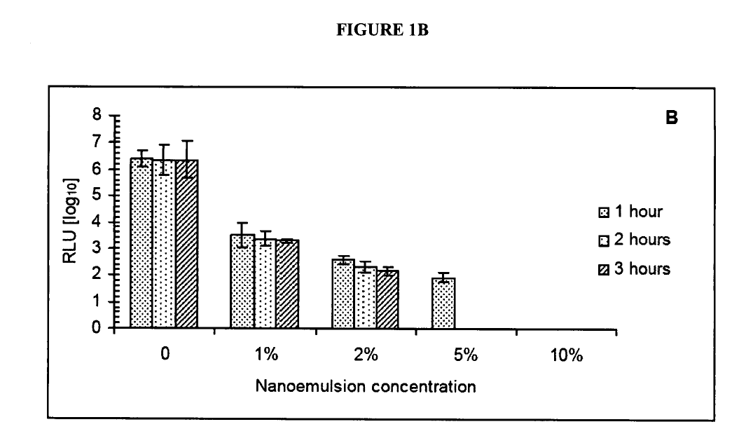

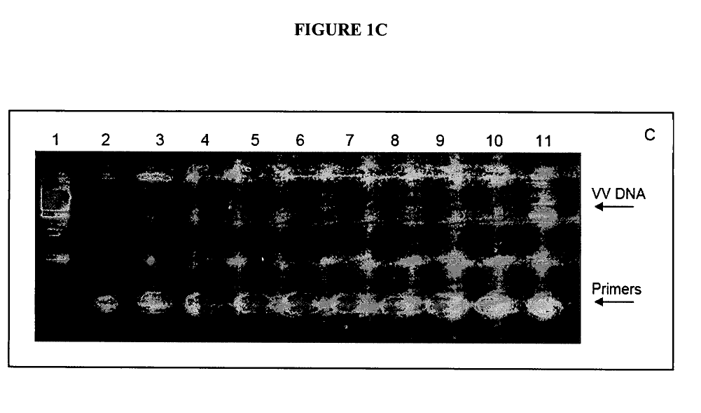

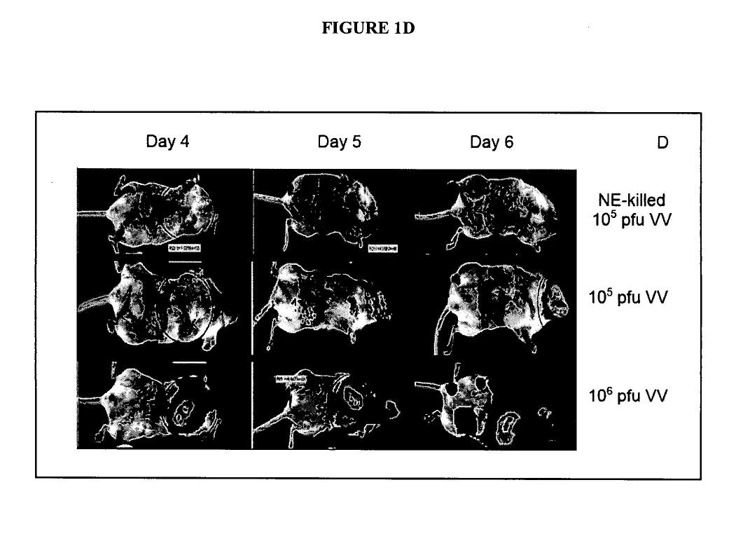

To evaluate virucidal activity of the NE in vitro, a range of NE concentrations was mixed with either VV_{WR} or VV_{WR-Luc} and incubated for 1 to 3 hours at 37° C. Results of both plaque reduction (PRA) and luciferase bioluminescence assays indicated NE concentration dependent inactivation of both viruses. The 10% NE completely inactivates greater than 10⁶ pfu of vaccinia within 1 hour of incubation (See FIGS. 1A and B). Subsequent passages of the culture supernatants from cells infected with VV inactivated with 10% NE showed no evidence of surviving virus.

Complete inactivation of virus in the NE preparations was further demonstrated in vivo after intranasal administration of inactivated VV_{WR} using PCR amplification of DNA isolated from mice lungs after administration. No viral DNA was detected in any of the treated mice (See FIG. 1C) while a control PCR (lung DNA spiked with VV_{WR} DNA) resulted in the product of the expected size >950 bp. In addition, in vivo bioluminescence imaging of mice also indicated an absence of viral infection and no evidence of virus amplification after administration of 10⁵ pfu of NE-inactivated VV_{WR-Luc}, as compared to a strong signal from mice nasally infected either 1×10⁵ or 1×10⁶ pfu of live VV_{WR-Luc} (See FIG. 1D). Thus, in vitro and in vivo results indicated that incubation with 10% NE for at least 60 minutes causes complete inactivation of VV. Accordingly, all virus inactivations were performed with 10% NE for 3 hours and subsequently diluted to 1% NE for immunization.

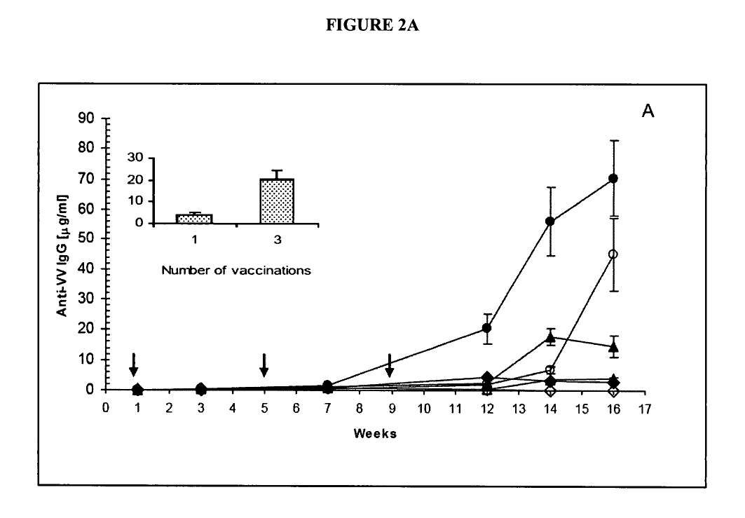

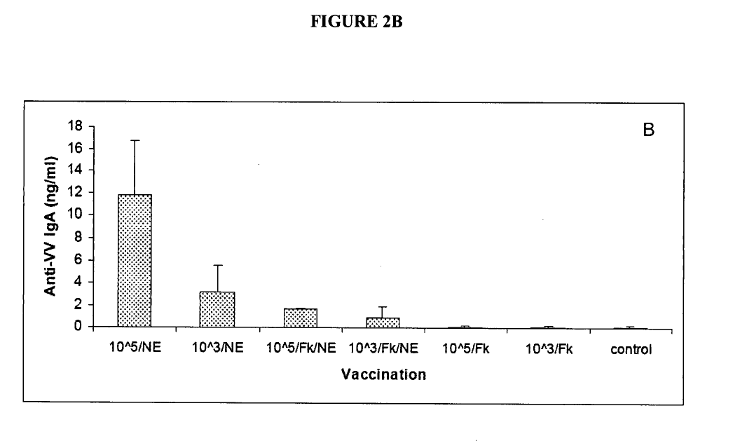

Next, experiments were designed to evaluate if compositions of the present invention (e.g., NE-killed VV) could produce protective immunity similar to that seen in humans vaccinated by scarification with live, replicating VV (See, e.g., Hammarlund et al., Nat. Med. 2003, 9; 1131-1137). Mice were intranasally (i.n.) immunized with six formulations containing either 10⁵ or 10³ pfu doses of VV_{WR} killed with NE (10⁵/NE and 10³/NE, respectively), formalin-killed virus mixed with 1% NE (10⁵/Fk/NE and 10³/Fk/NE, respectively), and formalin-killed virus in saline (10⁵/Fk and 10³/Fk). Control mice were treated with 1% NE alone. Antibody responses were characterized three weeks after initial vaccine administration (See FIG. 2). Immunity was boosted with subsequent administrations, at 5 and 9 weeks (FIG. 2A). Significant anti-VV IgG levels were detected after booster immunization in serum from mice vaccinated with either 10⁵/NE or 10⁵/NFk/NE with a mean anti-VV IgG concentrations of 1.5 μg/ml and 1 μg/ml, respectively. After a second boost, anti-VV antibody concentrations increased in all groups, and at the conclusion of the experiment (at 16 weeks), immunization with 10³/NE and 10⁵/NE, produced highest responses with mean concentrations of 44 μg/ml and 70 μg/ml of anti-VV IgG, respectively, followed by 10⁵/Fk/NE (17 μg/ml). Immunizations with 10³/Fk/NE, and either 10⁵/Fk or 10³/Fk formulations of vaccine consistently produced low levels of anti-VV antibodies, which did not increase significantly after booster administrations (See FIG. 2A). A comparison of a single-dose with a three-dose schedule of immunization with 10⁵/NE showed that a single dose of vaccine produced significant (˜4 μg/ml), albeit lower than three-dose, concentration of serum anti-VV IgG at 12 weeks after immunization. Thus, in some embodiments, the present invention provides that a single dose of VV/NE vaccine may be sufficient to initiate immune responses (e.g., mucosal or systemic immune responses), that can be enhanced by subsequent immunization (See FIG. 2A insert). No specific anti-VV antibodies were detected in any of the control mice.

Analysis of cross-reactive anti-VV antibodies indicated that serum IgG from mice immunized with NE-killed virus reacted with both formalin-crosslinked (alkylated) and with alcohol-fixed (denatured but not alkylated) viral proteins. Vaccination with VV/NE vaccine produced anti-VV IgG with 25 fold higher reactivity to the native, non-alkylated epitopes (similar to serum from mice exposed to a live virus), and those antibodies were also effective in recognizing formalin fixed viral proteins. In contrast, sera from animals vaccinated with formalin-killed virus, either alone or mixed with NE, did not have increased reactivity with native VV epitopes.

### EXAMPLE 3

### Subjects Administered Nanoemulsion-Killed Vaccinia Virus Possess Mucosal Immunity to Vaccinia Virus

Mucosal immunity was assayed by VV-specific secretory IgA antibody in bronchialalveolar fluids (BAL). Anti-VV IgA was detected in BAL from animals immunized with either 10³/NE or 10⁵/NE. Animals vaccinated with formulations containing formalin-killed virus, whether diluted in saline or NE, did not produce measurable mucosal response despite the presence of serum anti-VV IgG (See FIG. 2B). Thus, the present invention provides that a composition comprising NE-killed VV generates mucosal immunity in a subject (e.g., as demonstrated by the presence of VV-specific secretory IgA antibodies in the BAL of the subject) whereas compositions that do not contain NE-killed VV (e.g., formalin-killed VV) are not capable of generating mucosal immunity to VV.

### EXAMPLE 4

### Serum and Bronchial Alveolar Lavage (BAL) from Subjects Administered Nanoemulsion-Inactivated Vaccinia Virus Possess Virus-Neutralizing Antibodies

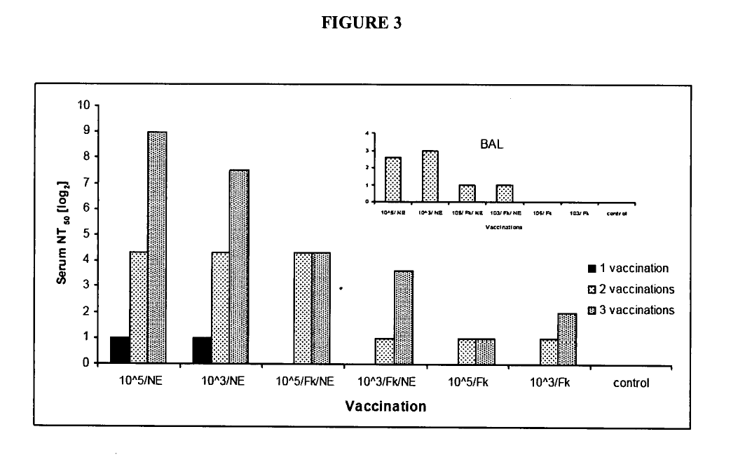

The biological relevance of the anti-VV antibody response was assessed in the virus neutralization assays. Neutralizing activity was detected in the serum of some mice after the single vaccination (FIG. 3A). However, consistent titers of serum neutralizing antibodies were present after two immunizations with either 10⁵/NE 10³/NE or 10⁵/Fk/NE. The mean 50% neutralization titer (NT₅₀) for each of these groups was ≧20. In contrast, animals vaccinated with 10³/Fk/NE, 10³/Fk or 10⁵/Fk, virus neutralization was observed only in the lowest serum dilution. Subsequent immunization produced greater than a ten fold increase in NT₅₀ titers, but only in the mice vaccinated with NE-killed virus (10³/NE and 10⁵/NE, NT₅₀=180 and NT₅₀=500, respectively). Third vaccination with any of formulations containing formalin-killed virus did not significantly increase VV neutralization. Significant neutralizing activity was also detected in BAL fluids from mice vaccinated with either 10³/NE or 10⁵/NE, and was lower in BAL from mice immunized with either 10³/Fk/NE or 10⁵/NFk/NE (See FIG. 3 insert). Neutralizing activity was absent in BAL of mice immunized with formalin-killed virus diluted in saline and in the control, not vaccinated animals. Thus, the present invention provides that despite inactivation (e.g. complete neutralization) of VV, nanoemulsions comprising inactivated VV of the present invention retain important immunogenic eptitopes (e.g., recognized and responded to by the immune system (e.g., humoral immune system) of a subject).

### EXAMPLE 5

### Comparison of Response to Native VV_{-WR} and VV_{-WR-Luc}

VV_{-WR-LUC} has identical surface proteins as the native strain, but expresses luciferase protein during infection. This allows for mortality assessment and monitoring of viral infection in challenged animals with imaging techniques. Comparison of antibodies in VV_{-WR} immunized animals versus both viral strains either in ELISA, Western blot or virus neutralization assays showed no difference between VV_{-WR} and VV_{-WR-Luc}.

### EXAMPLE 6

### Administration of NE-Killed VV Generates VV Specific Cellular Immune Responses

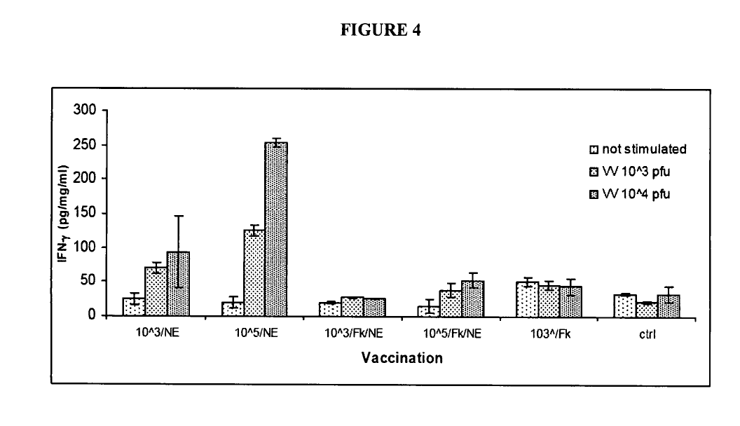

The effect of NE based vaccine on cellular response was explored using an in vitro cytokine expression assay in splenocytes. Individual cultures of mouse splenocytes were stimulated with 10³ and 10⁴ pfu of live vaccinia. VV-specific cellular immune responses were demonstrated by IFN-γ expression in vitro in splenocytes from animals immunized with either 10³/NE or 10⁵/NE. In contrast, no increase VV-specific IFN-γ production was observed in splenocytes from animals immunized with formalin-killed virus, even when was it was mixed with nanoemulsion. Production of IFN-γ in cells from mice treated with VV/NE vaccine indicates Th1 polarization of cellular response. No antigen specific cytokine expression was detected in control splenocyte cultures (See FIG. 4).

### EXAMPLE 7

### Subjects Administered NE-Killed VV are Protected Against Challenge with Live, Infectious VV

Protective immunity produced by mucosal immunization was evaluated in the challenge studies. Three groups of mice were nasally immunized with three doses of either 10⁵/NE, 10⁵/Fk/NE or 10⁵/Fk vaccine. Control animals were treated with saline. At 12 weeks mice were challenged with 10×LD₅₀ (2×10⁶ pfu) of live VV_{WR-Luc}. Body weight and temperature were measured two times a day and animals were imaged for VV_{WR-Luc} luminescence once a day. All 10⁵/NE vaccinated mice survived viral challenge (See FIG. 5A). Mice vaccinated with 10⁵/NFk/NE and 10⁵/Fk had 40% and 20% survival rates, respectively. Although not fully protective, vaccination with 10⁵/Fk/NE also extended mean time till death (TTD) from 5 to 7 days. None of the control mice survived challenge. Bioluminescence imaging used for assessment of viral infection demonstrated that two of the five 10⁵/NE immunized mice had minimally detectable virus replication which did not affect their weight and body temperature while the other three had more progressive replication that resolved within 6 days after challenge (See FIG. 5B). However, none of these animals had clinical evidence of infection. In contrast, all non-vaccinated controls became ill and died or were humanely euthanized within 4 to 7 days of virus challenge. These animals had massive virus replication and spreading of the infection throughout the nasopharyngeal passage, lung and abdomen as presented in photon flux data. In 10⁵/NE vaccinated mice, a low grade infection after i.n. challenge was limited to the head (nose) of vaccinated animals, without spreading to the chest and abdomen (See Table 1 below).

TABLE 1

| | |
|---|---|
| Vaccinated | Controls |

| | Days | avg | sem | avg | sem |
|---|---|---|---|---|---|
| Head | 2 | 7.0 | 3.3 | 33.8 | 12.0 |
| | 3 | 26.1 | 13.1 | 135.9 | 86.1 |
| | 4 | 75.2 | 16.5 | 431.0 | 252.0 |
| | 5 | 56.6 | 17.1 | 924.7 | 355.6 |
| Chest | 2 | 8.2 | 1.8 | 24.0 | 3.8 |
| | 3 | 13.3 | 2.0 | 119.8 | 25.0 |
| | 4 | 22.1 | 6.3 | 216.3 | 62.0 |
| | 5 | 16.6 | 1.3 | 618.2 | 135.8 |
| Abdomen | 2 | 12.1 | 1.0 | 19.2 | 5.1 |
| | 3 | 13.7 | 1.3 | 23.8 | 8.2 |
| | 4 | 23.0 | 2.8 | 28.3 | 10.0 |
| | 5 | 14.8 | 1.8 | 32.1 | 6.9 |

Taken together, the imaging studies suggested an inverse correlation between the dissemination of infection and survival. The presence of self-limiting infection in some immunized mice correlated with the levels of neutralizing antibodies in the individual animals.

To further investigate effectiveness of mucosal NE-based vaccine, the i.n. immunization with three doses of 10⁵/NE was compared with vaccination by scarification with live VV_{WR} (10⁵/sc). At 12 weeks mice were i.n. challenged with the escalating doses of live VV_{WR}. Survival data indicate that mucosal vaccination produced protective immunity equal to vaccination by scarification which is typically used for the human smallpox vaccine (See Table 2, below).

TABLE 2

| Challenge | | Survival^{a} | | |
|---|---|---|---|---|
| Dose [pfu] | ×LD50^{b} | NE vaccine | Scarification | Controls |
| 1.0.E+07 | 77 | 5/5 | 5/5 | 0/5 |
| 2.0.E+06 | 15 | 5/5 | 5/5 | 0/5 |
| 4.0.E+05 | 3 | 5/5 | 5/5 | 1/5 |
| 8.0.E+04 | 0.62 | 5/5 | 5/5 | 3/5 |
| 1.6.E+04 | 0.12 | 5/5 | 5/5 | 5/5 |
| 3.2.E+03 | 0.02 | 5/5 | 5/5 | 5/5 |

^{a}presented as a ratio of surviving to all mice
^{b}calculated 1 × LD₅₀ was 5.13 × 10⁵ pfu of VV_{WR}.

All mice vaccinated either with mucosal VV/NE vaccine or by scarification survived intranasal challenges with the maximal dose of 1×10⁷ pfu of VV_{WR} (77×LD₅₀). Index of protection against lethal challenge (IP_{LD}) was 1.9 for both the NE-based vaccine and scarification. All control non-vaccinated animals died after challenge with 15×LD₅₀ VV_{WR}. The high level of protection attained with i.n immunization was also seen in weight loss analysis of surviving mice. Although mucosal vaccination did not completely protect mice against respiratory infection with high doses of VV_{WR} (See Table 3, below), animals immunized with NE vaccine did not have clinical evidence of illness and had average weight loss of 10% or less, whereas surviving mice in control groups lost more than 25% of weigh at much lower doses of VV_{WR}. Statistical analysis indicated differences with p value <0.01 between body weight of immunized and control mice. Index of protection against infection (IP_{ID}) was 1 for VV/NE vaccine and 2.2 for scarification.

TABLE 3

| Challenge | | Protected mice^{a} | | |
|---|---|---|---|---|
| Dose [pfu] | ×LD₅₀^{b} | NE vaccine | Scarification | Controls |
| 1.0.E+07 | 77 | 0/5 | 3/5 | 0/5 |
| 2.0.E+06 | 15 | 0/5 | 5/5 | 0/5 |
| 4.0.E+05 | 3 | 1/5 | 5/5 | 0/5 |
| 8.0.E+04 | 0.62 | 2/5 | 5/5 | 0/5 |
| 1.6.E+04 | 0.12 | 4/5 | 5/5 | 0/5 |
| 3.2.E+03 | 0.02 | 5/5 | 5/5 | 5/5 |

^{a}presented as a ratio of mice which did not have decrease in body weight and temperature at any time after challenge to all mice
^{b}calculated 1 × LD₅₀ was 5.13 × 10⁵ pfu of VV_{WR}.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of inducing an immune response to an orthopox virus in a subject comprising:
a) providing a composition comprising a nanoemulsion and an immunogen, wherein said immunogen comprises an orthopox virus inactivated by said nanoemulsion; and
b) administering said composition to said subject under conditions such that said subject generates an immune response to said orthopox virus.

2. The method of claim 1, wherein said administering comprises contacting a mucosal surface of said subject with said composition.

3. The method of claim 2, wherein said mucosal surface comprises nasal mucosa.

4. The method of claim 1, wherein said inducing an immune response induces immunity to said orthopox virus in said subject.

5. The method of claim 4, wherein said immunity comprises systemic immunity.

6. The method of claim 4, wherein said immunity comprises mucosal immunity.

7. The method of claim 1, wherein said immune response comprises increased expression of IFN-γ in said subject.

8. The method of claim 1, wherein said immune response comprises a systemic IgG response to said inactivated orthopox virus.

9. The method of claim 1, wherein said immune response comprises a mucosal IgA response to said inactivated orthopox virus.

10. The method of claim 1, wherein said orthopox virus inactivated by said nanoemulsion is administered to said subject under conditions such that between 10 and 10³ pfu of said inactivated virus is present in a dose administered to said subject.

11. The method of claim 1, wherein a 10% nanoemulsion solution is utilized to inactivate said vaccinia virus.

12. The method of claim 1, wherein said nanoemulsion comprises W₂₀5EC.

13. The method of claim 1, wherein said immunity protects said subject from displaying signs or symptoms of disease caused by said orthopox virus.

14. The method of claim 1, wherein said immunity protects said subject from challenge with a subsequent exposure to live orthopox virus.

15. The method of claim 1, wherein said composition further comprises an adjuvant.

16. The method of claim 1, wherein said subject is a human.

17. The method of claim 1, wherein said orthopox virus is vaccinia virus.

18. The method of claim 13, wherein said subject is protected from displaying signs and symptoms of smallpox.

19. A composition for stimulating an immune response comprising a nanoemulsion and an orthopox virus inactivated by said nanoemulsion, wherein said composition is configured to induce immunity to said orthopox virus in a subject.

20. The composition of claim 19, wherein said nanoemulsion comprises W₂₀5EC.

21. The composition of claim 19, wherein said composition provides said subject between 10 and 10³ pfu of said inactivated virus when administered to said subject.

22. The composition of claim 19, wherein a dose of said composition administered to said subject comprises a 1% nanoemulsion solution.

23. The composition of claim 19, wherein said inactivated orthopox virus is heat stable in said nanoemulsion.

24. The composition of claim 23, wherein said orthopox virus is stable for greater than four weeks in said nanoemulsion.

25. The composition of claim 19, wherein said composition is diluted prior to administration to a subject.

26. The composition of claim 23, wherein said subject is a human.

27. The composition of claim 19, wherein said immunity is systemic immunity.

28. The composition of claim 19, wherein said immunity is mucosal immunity.

29. The composition of claim 19, wherein said composition further comprises an adjuvant.

30. The composition of claim 19, wherein said orthopox virus is vaccinia virus.

31. The composition of claim 19, wherein said orthopox virus is selected from the group consisting of variola virus, cowpox, monkeypox, gergilpox, and camelpox.

32. A kit comprising a composition for stimulating an immune response comprising a nanoemulsion and an orthopox virus inactivated by said nanoemulsion, wherein said composition is configured to induce immunity to said orthopox virus in a subject, and instructions for administering said composition.

33. The kit of claim 32, wherein said orthopox virus is vaccinia virus.

34. The kit of claim 32, wherein said orthopox virus is selected from the group consisting of variola virus, cowpox, monkeypox, gerbilpox, and camelpox.
